(19) United States
(12) Patent Application Publication
(10) Pub. No.: US 2022/0365099 A1
(43) Pub. Date: Nov. 17, 2022

(54) DERIVATIZATION OF BETA-LACTAM ANTIBIOTICS FOR MASSSPEC MEASUREMENTS IN PATIENT SAMPLES

(71) Applicant: Roche Diagnostics Operations, Inc., Indianapolis, IN (US)

(72) Inventor: Gaston Hubertus Maria Vondenhoff, Penzberg (DE)

(21) Appl. No.: 17/745,429

(22) Filed: May 16, 2022

Related U.S. Application Data
(63) Continuation of application No. PCT/EP2020/081818, filed on Nov. 12, 2020.

(30) Foreign Application Priority Data
Nov. 15, 2019 (EP) 19209516.4

(51) Int. Cl. G01N 33/68; G01N 33/543; B01L 3/00

(52) U.S. Cl. CPC G01N 33/6854; G01N 33/54326; B01L 3/5082; B01L 2200/16

(57) ABSTRACT

The present invention relates to derivatization of antibiotic analytes as well as methods of determining the amount or concentration of derivatized antibiotic analytes in an obtained sample.

The present invention relates to derivatization of antibiotic analytes as well as methods of determining the amount or concentration of derivatized antibiotic analytes in an obtained sample.

### BACKGROUND

β-lactam antibiotics are a class of antibiotics that are prescribed most commonly as either specific or broad-spectrum antibiotics for patients infected with bacteria. This class of antibiotics works by interfering with the crosslinking of the peptidoglycan layer that is most dominant in Gram-positive bacteria. They exhibit a bacteriocidal effect, which is concentration dependent. Therefore, it is critical to keep the antibiotic concentration above the MIC. However, higher concentrations lead to adverse effects. Moreover, it has been reported that the pharmacokinetics of these compounds is highly variable and therefore unpredictable (Ronilda D'Cunha et al.; 2018; Antimicrobial Agents and Chemotherapy 62 (9)).

The mechanism of action of these antibiotics is by reacting the four-membered β-lactam ring with the D-alanyl-D-alanyl-transpeptidase, thereby inhibiting the formation of cross-links between the peptidoglycan polymers of the outer cell-wall.

Thus, the relative instable lactam moiety is responsible for the mechanism of action of these antibiotics. However, this instability also leads to a partial hydrolyzation of these compounds upon dissolution in protic solvents. Even more so, hydrolyzation is naturally further catalyzed by the presence of acid or base and enhanced with elevated temperatures. Obviously, hydrolyzed antibiotics are no longer active compounds that can inhibit bacterial growth.

Therapeutic Drug Monitoring (TDM) is a field of medicine that aims to quantify drugs from human sample material with the aim to monitor drug concentrations in the body. Considerable efforts have been made to study and address β-Lactam instability in the field of Therapeutic Drug Monitoring (TDM), mostly focusing storage conditions that aim to retain the compounds in their native (i.e. unhydrolyzed) form (Zander et al.; 2016; Clinical Chemistry and laboratory Medicine; 54(2)). As hydrolyzation continues after patient sampling (e.g. blood collection), obtaining accurate concentrations of the native β-Lactam antibiotics in the patient is currently very challenging. Since it is crucial to carefully monitor antibiotic concentrations, a valid and stable method to quantify these compounds from human and animal matrices is required.

### SUMMARY OF THE INVENTION

In a first aspect, the present invention relates to an (automated) method of determining the amount or concentration of one or more derivatized antibiotic analytes in an obtained sample comprising
a) optionally pre-treating and/or enriching the sample, in particular using magnetic beads, and
b) determining the amount or concentration of the one or more antibiotic analyte in the sample.

In a second aspect, the present invention relates to an (automated) method of determining the amount or concentration of one or more antibiotic analytes in an obtained sample, comprising
a) pre-treating the sample with a derivatization reagent, wherein the derivatization reagent comprises a nucleophile,
b) optionally enriching the sample obtained after step a), in particular using magnet beads, and
c) determining the amount or concentration of the one or more antibiotic analyte in the pre-treated sample obtained after step a) or after the optional enrichment step b).

In a third aspect, the present invention relates to an (automated) analytical system (in particular LC/MS system) adapted to perform the method of the first or second aspect.

In a fourth aspect, the present invention relates to a sampling tube for collecting a patient, sample comprising a nucleophilic derivatization reagent suitable to stabilize one or more antibiotic analytes in a sample.

In a fifth aspect, the present invention relates to the use of a nucleophilic derivatization reagent for determining the amount or concentration of one or more antibiotic analytes in a sample.

In a sixth aspect, the present invention relates to the use of a nucleophilic derivatization reagent to stabilize an antibiotic analyte in a sample of interest.

In a seventh aspect, the present invention relates to an antibiotic analyte stabilized by nucleophilic derivatization reagent.

### LIST OF FIGURES

FIG. 1: Schematic drawing of hydrolyzation pathway of Piperacillin.

FIG. 2: Measured peak area over 16 h of A) native Piperacillin (compound 5); and B) single hydrolyzed Piperacillin (9a or 9b) in water at room temperature. Depicted with confidence fit and P-test. For clarity, reference lines have been drawn through the average area values.

FIG. 3: Schematic drawing of derivatization reaction of Meropenem with different reagents: A) propylamine; B) butylamine, C) pentylamine.

FIG. 4. Schematic drawing of derivatization reaction of Piperacillin with different reagents: A) propylamine; B) butylamine, C) pentylamine.

FIG. 5: Measured peak area over 16 h of double derivatized Piperacillin (compound 7) in water at room temperature, for two MRM transitions. Depicted with confidence fit and F-test. For clarity, reference lines have been drawn through the average area values.

FIG. 6: Measured Peak Areas of Meropenem derivatised with reagents propylamine, butylamine, and pentylamine at different reaction conditions.

FIG. 7: Measured Peak Areas of Piperazilin derivatised with reagents propylamine, butylamine, and pentylamine at different reaction conditions.

FIG. 8: It is shown a schematic representation of a signal vs. concentration. The result of this is that the spiked concentration is higher than the actual concentration, calibration offset resulting from a difference of the spiked concentration than the actual concentration as shown in Example 4.

FIG. 9: Difference in area ratio between samples in neat and from serum for four concentrations as shown in Example 4.

FIG. 10: Precision and accuracy results of Example 5.

FIG. 11: Correlation calculated concentrations from both methods as shown in Example 5.

FIG. 12: Correlation calculated concentrations from both methods as shown in Example 5.

FIG. 13: Difference in accuracy between the two methods per replicate as shown in Example 5.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “measurement”, “measuring” or “determining” preferably comprises a qualitative, a semi-quantitative or a quantitative measurement.

The term “automated” refers to methods or processes which are operated largely by automatic equipment, i.e. which are operate by machines or computers, in order to reduce the amount of work done by humans and the time taken to do the work. Thus, in an automated method, tasks that were previously performed by humans, are now performed by machines or computers. Typically, the users only need to configure the tool and define the process. The skilled person is well aware that at some minor points manual intervention may still be required, however the large extend of the method is performed automatically.

In the context of the present disclosure, the term “analyte”, “analyte molecule”, or “analyte(s) of interest” are used interchangeably, referring to the chemical species to be analysed. Chemical species suitable to be analysed, i.e. analytes, can be any kind of molecule present in a living organism, include but are not limited to nucleic acid, amino acids, peptides, proteins, fatty acids, lipids, carbohydrates, steroids, ketosteroids, secosteroids molecules. Analytes may also be any substance that has been internalized by the organism, such as but not limited to therapeutic drugs, drugs of abuse, toxin, or a metabolite of such a substance. Therapeutic drugs include antibiotics, i.e. “antibiotic analytes”. Antibiotics are substance active against microbial organisms. Antibiotics are commonly classified based on their mechanism of action, chemical structure, or spectrum of activity. One class of antibiotics are β-lactam antibiotics. β-lactam antibiotics (beta-lactam antibiotics) are all antibiotic agents that contain a beta lactam ring in their molecular structures. These include but are not limited to penicillin derivatives (penams), cephalosporins (cephems), monobactams, carbapenems and carbacephems. Most β-lactam antibiotics work by inhibiting cell wall biosynthesis in the bacterial organism and are the most widely used group of antibiotics. The effectiveness of these antibiotics relies on their ability to reach the PBP intact and their ability to bind to the penicillin binding proteins (PPP).

Analytes may be present in a sample of interest, e.g. a biological or clinical sample. The terms “sample” or “sample of interest” are used interchangeably herein, referring to a part or piece of a tissue, organ or individual, typically being smaller than such tissue, organ or individual, intended to represent the whole of the tissue, organ or individual. Upon analysis, a sample provides information about the tissue status or the health or diseased status of an organ or individual. Examples of samples include but are not limited to fluid samples such as blood, serum, plasma, synovial fluid, spinal fluid, urine, saliva, and lymphatic fluid, or solid samples such as dried blood spots and tissue extracts. Further examples of samples are cell cultures or tissue cultures.

In the context of the present disclosure, the sample may be derived from an “individual” or “subject”. Typically, the subject is a mammal. Mammals include, but are not limited to, domesticated animals (e.g., cows, sheep, cats, dogs, and horses), primates (e.g., humans and non-primates such as monkeys), rabbits, and rodents (e.g., mice and rats).

Before being analysed, a sample may be pre-treated in a sample- and/or analyte specific manner. In the context of the present disclosure, the term “pre-treatment” refers to any measures required to allow for the subsequent analysis of a desired analyte. Pre-treatment measures typically include but are not limited to the elution of solid samples (e.g. elution of dried blood spots), addition of hemolizing reagent (HR) to whole blood samples, and the addition of enzymatic reagents to urine samples. Also the addition of internal standards (ISTD) is considered as pre-treatment of the sample.

Typically, an internal standard (ISTD) is a known amount of a substance which exhibits similar properties as the analyte of interest when subjected to the mass spectrometric detection workflow including any pre-treatment, enrichment and actual detection step). Although the ISTD exhibits similar properties as the analyte of interest, it is still clearly distinguishable from the analyte of interest. Exemplified, during chromatographic separation, such as gas or liquid chromatography, the ISTD has about the same retention time as the analyte of interest from the sample. Thus, both the analyte and the ISTD enter the mass spectrometer at the same time. The ISTD however, exhibits a different molecular mass than the analyte of interest from the sample. This allows a mass spectrometric distinction between ions from the ISTD and ions from the analyte by means of their different mass/charge (m/z) ratios. Both are subject to fragmentation and provide daughter ions. These daughter ions can be distinguished by means of their m/z ratios from each other and from the respective parent ions. Consequently, following calibration, a separate determination and quantification of the signals from the ISTD and the analyte can be performed. Since the ISTD has been added in known amounts, the signal intensity of the analyte from the sample can be attributed to a specific quantitative amount of the analyte. Thus, the addition of an ISTD allows for a relative comparison of the amount of analyte detected, and enables unambiguous identification and quantification of the analyte(s), of interest present in the sample when the analyte(s) reach the mass spectrometer. Typically, but not necessarily, the ISTD is an isotopically labeled variant (comprising e.g. ²H, ¹³C, or ¹⁵N etc. label) of the analyte of interest.

The term “immunoglobulin (Ig)” as used herein refers to immunity conferring glycoproteins of the immunoglobulin superfamily “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: α, γ, δ, ε, and μ. The type of heavy chain present defines the class of antibody, i.e. these chains are found IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al, (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgGI, 2, 3, and 4), named in order of their abundance in serum with IgGI being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CHI, CH2, CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCyI-Cy2-Cy3), whereas the human IgG light chain is composed of two immunoglobulin domains linked from N- to C-terminus in the order VL-CL, being either of the kappa or lambda type (VK-CK or VA.-CA.). Exemplified, the constant chain of human IgG comprises 447 amino acids. Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5^{th} ed. U.S. Department of Health and Human Service, National Institutes of Health, Bethesda, Md. The “EU index as in Kabat” refers to the residue numbering of the human IgG IEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-447 according to the EU index as in Kabat.

The terms “full-length antibody”, “intact antibody”, and “whole antibody” are used herein interchangeably to refer to an antibody in its substantially intact form, not antibody fragments as defined below. The terms particularly refer to an antibody with heavy chains that contain an Fc region.

Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al, (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA), “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

Accordingly, the term “antibody fragments” refers to a portion of an intact antibody, preferably comprising the antigen-binding region thereof. Antibody fragments include but are not limited to Fab, Fab′, F(ab′)₂, Fv fragments; diabodies; sdAb, nanobodies, scFv, di-scFvs, tandem scFvs, triabodies, diabodies, scDb, BiTEs, and DARTS.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

“Sandwich immunoassays” are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g. an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight, if more convenient) and under suitable conditions (e.g., from room temperature to 40° C. such as between 25° C. and 37° C. inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

An extremely versatile alternative sandwich assay format includes the use of a solid phase coated with the first partner of a binding pair, e.g. paramagnetic streptavidin-coated microparticles. Such microparticles are mixed and incubated with an analyte-specific binding agent bound to the second partner of the binding pair (e.g. a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay may include one or more washing step(s).

The term “detectably labeled” encompasses labels that can be directly or indirectly detected.

Directly detectable labels either provide a detectable signal or they interact with a second label to modify the detectable signal provided by the first or second label, e.g., to give FRET (fluorescence resonance energy transfer). Labels such as fluorescent dyes and luminescent (including chemiluminescent and electrochemiluminescent) dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling. In one embodiment detectably labeled refers to a label providing or inducible to provide a detectable signal, i.e. to a fluorescent label, to a luminescent label (e.g. a chemiluminescent label or an electrochemiluminescent label), a radioactive label or a metal-chelate based label, respectively.

Numerous labels (also referred to as dyes) are available which can be generally grouped into the following categories, all of them together and each of them representing embodiments according the present disclosure:

(a) Fluorescent Dyes

Fluorescent dyes are e.g. described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oreg., USA) and Pierce Biotechnology, Inc. (Rockford, Ill.).

(b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bay)3]2+ (which releases a photon at ˜620 nm) regenerating with TPA (Tripropylamine) in liquid phase or liquid-solid interface are used as ECL-labels.

Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment.

Such electrochemiluminescense is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605, 5,591,581, 5,597,910, PCT published application WO90/05296, PCT published application WO92/14139, PCT published application WO90/05301, PCT published application WO96/24690, PCT published application US95/03190, PCT application US97/16942, PCT published application US96/06763, PCT published application WO95/08644, PCT published application WO96/06946, PCT published application WO96/33411, PCT published application WO87/06706, PCT published application WO96/39534, PCT published application WO96/41175, PCT published application WO96/40978, PCT/US97/03653 and U.S. patent application Ser. No. 08/437,348 (U.S. Pat. No. 5,679,519). Reference is also made to a 1994 review of the analytical applications of ECL by Knight, et al. (Analyst, 1994, 119: 879-890) and the references cited therein. In one embodiment the method according to the present description is practiced using an electrochemiluminescent label.

Recently also iridium-based ECL-labels have been described (WO2012107419).

(c) Radioactive labels make use of radioisotopes (radionuclides), such as 3H, 11C, 14C, 18F, 32P, 35S, 64Cu, 68Gn, 86Y, 89Zr, 99TC, 111In, 123I, 124I, 125I, 131I, 133Xe, 177Lu, 211At, or 131Bi.

(d) Metal chelate complexes suitable as labels for imaging and therapeutic purposes are, well-known in the art (US 2010/0111861; U.S. Pat. Nos. 5,342,606; 5,428,155; 5,316,757; 5,480,990; 5,462,725; 5,428,139; 5,385,893; 5,739,294; 5,750,660; 5,834,461; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

The term “Mass Spectrometry” (“Mass Spec” or “MS”) relates to an analytical technology used to identify compounds by their mass. MS is a methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). The term “ionization” or “ionizing” refers to the process of generating an analyte ion having a net electrical charge equal to one or more electron units. Negative ions are those having a net negative charge of one or more electron units, while positive ions are those having a net positive charge of one or more electron units. The MS method may be performed either in “negative ion mode”, wherein negative ions are rated and detected, or in “positive ion mode” wherein positive ions are generated and detected.

“Tandem mass spectrometry” “MS/MS” involves multiple steps of mass spectrometry selection, wherein mentation of the analyte occurs in between the stages. In a tandem mass spectrometer, ions are formed in the ion source and separated by mass-to-charge ratio in the first stage of mass spectrometry (MS1). Ions of a particular mass-to-charge ratio (precursor ions or parent ion) are selected and fragment ions (or daughter ions) are created by collision-induced dissociation, ion-molecule reaction, or photodissociation. The resulting ions are then separated and detected in a second stage of mass spectrometry (MS2).

Most sample workflows in MS further include sample preparation and/or enrichment steps, wherein e.g. the analyte(s) of interest are separated from the matrix using e.g. gas or liquid chromatography. Typically, for the mass spectrometry measurement the following three steps are performed:
1. a sample comprising an analyte of interest is ionized, usually by adduct formation with cations, often by protonation to cations. Ionization source include but are not limited to electrospray ionization (ESI) and atmospheric pressure chemical ionization (APCI).
2. the ions are sorted and separated according to their mass and charge. High-field asymmetric-waveform ion-mobility spectrometry (FAIMS) may be used as ion filter.
3. the separated ions are then detected, e.g. in multiple reaction mode (MRM), and the results are displayed on a chart.

The term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

The term “atmospheric pressure chemical ionization” or “APCI,” refers to mass spectrometry methods that are similar to ESI; however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated N₂ gas may be used to improve removal of solvent. The gas-phase ionization in APCI can be more effective than ESI for analyzing less-polar entity.

“Multiple reaction mode” or “MRM” is a detection mode for a MS instrument in which a precursor on and one or more fragment ions are selectively detected.

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

Mass spectrometric determination may be combined with additional analytical methods including chromatographic methods such as gas chromatography (GC), liquid chromatography (LC), particularly HPLC, and/or ion mobility-based separation techniques.

The term “chromatography” refers to a process in which a chemical mixture carried by a liquid or gas is separated into components as a result of differential distribution of the chemical entities as they flow around or over a stationary liquid or solid phase.

The term “liquid chromatography” or “LC” refers to a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Methods in which the stationary phase is more polar than the mobile phase (e.g., toluene as the mobile phase, silica as the stationary phase) are termed normal phase liquid chromatography (NPLC) and methods in which the stationary phase is less polar than the mobile phase (e.g., water-methanol mixture as the mobile phase and C18 (octadecylsilyl) as the stationary phase) is termed reversed phase liquid chromatography (RPLC).

“High performance liquid chromatography” or “HPLC” refers to a method of liquid chromatography in which the degree of separation is increased by forcing the mobile phase under pressure through a stationary phase, typically a densely packed column. Typically, the column is packed with a stationary phase composed of irregularly or spherically shaped particles, a porous monolithic layer, or a porous membrane. HPLC is historically divided into two different sub-classes based on the polarity of the mobile and stationary phases. Methods in which the stationary phase is more polar than the mobile phase (e.g., toluene as the mobile phase, silica as the stationary phase) are termed normal phase liquid chromatography (NPLC) and the opposite (e.g., water-methanol mixture as the mobile phase and C18 (octadecylsilyl) as the stationary phase) is termed reversed phase liquid chromatography (RPLC). Micro LC refers to a HPLC method using a column having a narrow inner column diameter, typically below 1 mm, e.g. about 0.5 mm. “Ultra high performance liquid chromatography” or “UHPLC” refers to a HPLC method using a pressure of 120 MPa (17,405 lbf/in2), or about 1200 atmospheres. Rapid LC refers to an LC method using a column having an inner diameter as mentioned above, with a short length <2 cm, e.g. 1 cm, applying a flow rate as mentioned above and with a pressure as mentioned above (Micro LC, UHPLC). The short Rapid LC protocol includes a trapping/wash/elution step using a single analytical column and realizes LC in a very short time <1 min.

Further well-known LC modi include Hydrophilic interaction chromatography (HILIC) size-exclusion LC, ion exchange LC, and affinity LC.

LC separation may be single-channel LC or multi-channel LC comprising a plurality of LC channels arranged in parallel. In LC analytes may be separated according to their polarity or log P value, size or affinity, as generally known to the skilled person.

In the context of the present invention, the term “complex” refers to a chemical substance having a specific chemical structure. Said complex may comprise one or more functional units. Each unit may fulfil a different functionality, or two or more functional units may fulfil the same function.

In the context of the present invention, the term “nucleophile” refers to a chemical species that donates an electron pair to form a chemical bond. Nucleophiles that exists in a water medium include but are not limited to —NH₂, —OH, —SH, —Se, (R′,R″,R′″)P, Na₃—, RCOOH, F—, Cl—, Br—, I—. In the context of the present invention, the term “nucleophilic derivatization reagent” or “nucleophile derivatization reagent” refers to reagents comprising such nucleophile. A nucleophilic derivatization reagent comprises a moiety, carrying an orbital that serves as the highest occupied molecular orbital (HOMO) that is able to attack the lowest unoccupied molecular orbital (LUMO) of the substance of interest, such as an analyte of interest, thereby forming a new molecule comprised of the formerly nucleophilic unit and the analyte moiety.

A “kit” is any manufacture (e.g. a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in viva or in vitro use. The computer program code may be provided on a data storage medium or device such as an optical storage medium (e.g., a Compact Disc) or directly on a computer or data processing device. Moreover, the kit may, comprise standard amounts for the biomarkers as described elsewhere herein for calibration purposes.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

The term “sampling tube” or “sample collection tube” refers to any device with a reservoir appropriate for receiving a blood sample to be collected.

### EMBODIMENTS

Commonly used approaches to measure antibiotics, in particular β-lactam antibiotics, aim to defuse their instability. In contrast, the present invention does not defuse but employs the reactivity of the antibiotics by reacting them with a suitable nucleophile and thereby providing accurate measurements of antibiotics in patient samples.

In a first aspect, the present invention relates to a method of determining the amount or concentration of one or more derivatized antibiotic analytes in an obtained sample comprising
a) optionally pre-treating and/or enriching the sample, in particular using magnetic beads, and
b) determining the amount or concentration of the one or more antibiotic analyte in the sample.

In embodiments, the derivatized antibiotic analyte is an adduct formed of a nucleophilic derivatization reagent and an antibiotic analyte. In particular embodiments, the derivatized antibiotic analyte is a covalent adduct formed of a nucleophilic derivatization reagent and an antibiotic analyte. In embodiments, the derivatized antibiotic analyte exhibits an increased stability in comparison to the same underivatized antibiotic analyte.

In embodiments, the antibiotic analyte is a lactam antibiotic analyte. In embodiments, the antibiotic analyte is a β-lactam antibiotic analyte. In particular embodiments, the antibiotic analytes is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.

In embodiments, the antibiotic analyte is derivatized with a nucleophilic derivatization reagent, in particular a reagent comprising, an amine group, in particular a primary or secondary amine, in particular a primary amine group. A primary amine group has the advantage that the incubation time can be reduced in comparison to a secondary amine. In embodiments, the antibiotic analyte is derivatized with a nucleophilic derivatization reagent comprises more than 3 C-atoms, in particular 3 to 20 C-atoms, in particular 3 to 10 C-atoms, in particular 3-5 C-atoms, in particular 4 C-atoms. In embodiments, the antibiotic analyte is derivatized with a linear or branched nucleophilic derivatization reagent, in particular with a linear amine, in particular with a linear primary amine, in particular with a linear primary amine comprising 3 to 5 C-atoms. In embodiments, the antibiotic analyte is derivatized with a nucleophilic derivatization reagent selected from the group consisting of propylamine, butylamine, or pentylamine, in particular primary linear butylamine or primary linear pentylamine. Thus, MS interferences can be reduced or avoided.

In embodiments, the derivatized antibiotic analyte is, derivatized in at least one of its chemical moieties. The person skilled in the art of chemistry is well aware of chemical moieties which are suitable to be derivatized, in particular with a nucleophilic derivatization reagent. In particular embodiments, the derivatized antibiotic analyte is derivatized in one, two or three of its chemical moieties.

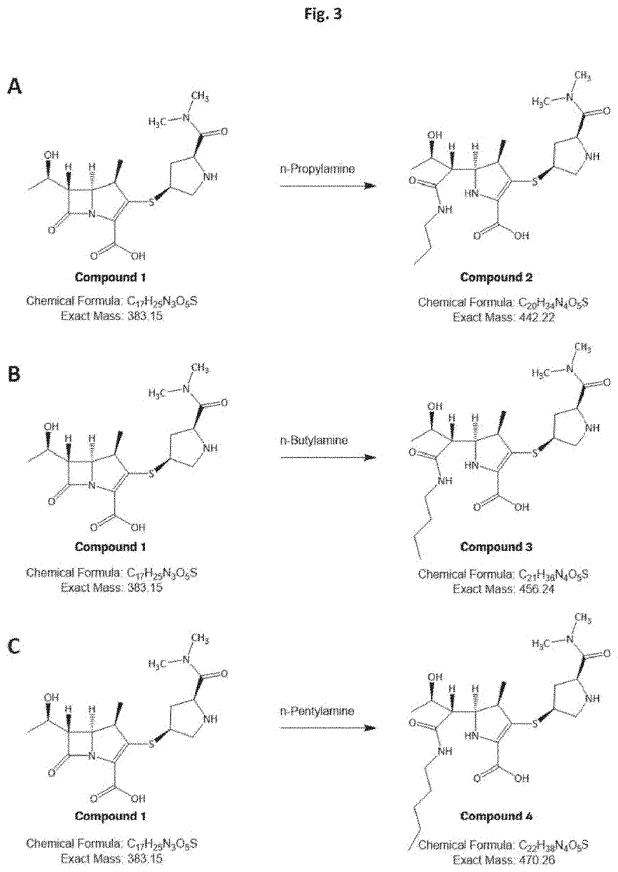

In particular embodiments, wherein the antibiotic analyte is Meropenem, it is derivatized with a nucleophilic derivatization reagent comprising butylamine. See also FIG. 3

In particular embodiments, wherein the antibiotic analyte is Piperacillin, it is derivatized with a nucleophilic derivatization reagent comprising pentylamine

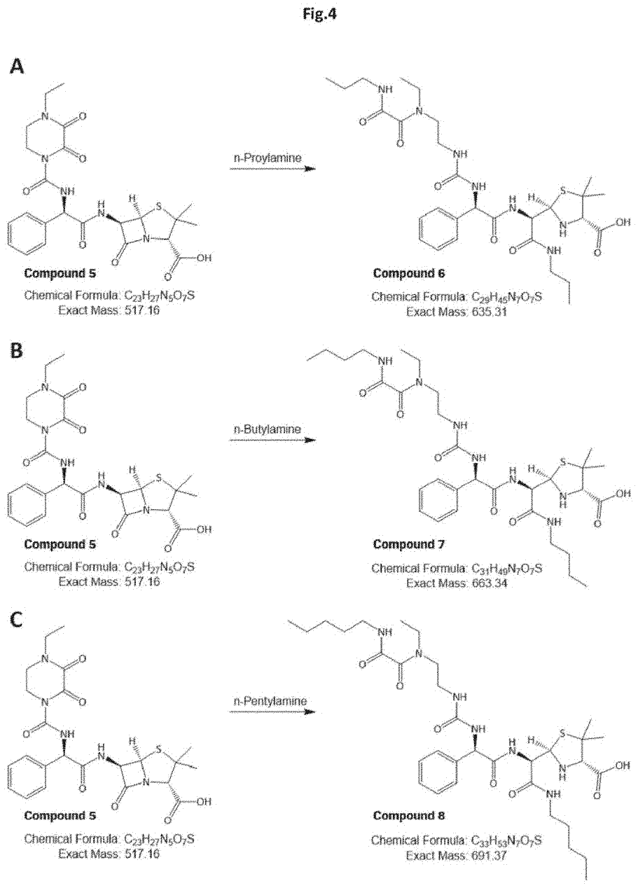

In particular embodiments, wherein the antibiotic analyte is Piperacilin, it is derivatized with a nucleophilic derivatization reagent comprising pentylamine at two of its chemical moieties, in particular at the β-lactam ring and at the piperazine ring. See also FIG. 4

In embodiments, the samples comprising a derivatized antibiotic analyte may be pre-treated and/or enriched by various methods. The pre-treatment method is dependent upon the type of sample, such as blood (fresh or dried), plasma, serum, urine, or saliva, whereas the enrichment method is dependent on the analyte of interest. It is well known to the skilled person which pre-treatment method is suitable for which sample type. It is also well-known to the skilled person which enrichment method is suitable for which analyte of interest.

In embodiments, wherein the sample is a whole blood sample, it is assigned to one of two pre-defined sample pre-treatment (PT) workflows, both comprising the addition of an internal standard (ISTD) and a hemolysis reagent (HR) followed by a pre-defined incubation period (Inc), where the difference between the two workflows is the order in which the internal standard (ISTD) and a hemolysis reagent (HR) are added. In embodiments, the ISTD is added first to the obtained sample followed by the addition of the hemolysis reagent. In embodiments, the ISTD is added to the obtained sample subsequent to the addition of the hemolysis reagents. In embodiments, water is added as a hemolysis reagents, in particular in an amount of 0.5:1 to 20:1 mL water/mL sample, in particular in an amount of 1:1 to 10:1 mL water/mL sample, in particular in an amount of 2:1 to 5:1 mL water/mL sample.

In embodiments, wherein the sample is a urine sample, it is assigned to one of other two pre-defined sample PT workflows, both comprising the addition of an ISTD and an enzymatic reagent followed by a pre-defined incubation period, where the difference between the two workflows is the order in which the internal standard and an enzymatic reagent are added. In embodiments, the ISTD is added first to the obtained sample followed by the addition of the enzymatic reagent. In embodiments, the ISTD is added to the obtained sample subsequent to the addition of the enzymatic reagents. An enzymatic reagent is typically a reagent used for glucuronide cleavage or protein cleavage or any pre-processing of analyte or matrix. In embodiments, the enzymatic reagent in selected from the group consisting of glucuronidase, (partial) exo- or endo-deglycoslation enzymes, or exo- or endo proteases. In embodiments, glucoronidase is added in amount of 0.5-10 mg/ml, in particular in an amount of 1 to 8 mg/ml, in particular in an amount of 2 to 5 mg/ml.

In embodiments, wherein the sample is plasma or serum it is assigned to another pre-defined PT workflow including only the addition of an internal standard (ISTD) followed by a pre-defined incubation time.

It is well-known to the skilled person which incubation time and temperature to choose for a sample treatment, chemical reaction or method step considered and as named herein above or below. In particular, the skilled person knows that incubation time and temperature depend upon each other, in that e.g. a high temperature typically leads to a shorter incubation period and vice versa.

The (pre-treated) sample may be further subjected to at least one enrichment workflow. The enrichment workflow may include one or more enrichment methods. Enrichment methods are well-known in the art and include but are not limited to chemical enrichment methods including but not limited to chemical precipitation, and enrichment methods using solid phases including but not limited to solid phase extraction methods, bead workflows, and chromatographic methods (e.g. gas or liquid chromatography).

In embodiments, a first enrichment workflow comprises the addition of a solid phase, in particular of solid beads, carrying analyte-selective groups, to the (pre-treated) sample. In embodiments, a first enrichment workflow comprises the addition of magnetic or paramagnetic heads carrying analyte-selective groups to the pre-treated sample.

In embodiments, the magnetic beads comprise a magnetic core coated with a styrene based polymer that is hypercrosslinked via Friedel-Crafts alkylation and further modified with addition of —OH groups.

In embodiments, the magnetic beads comprise a magnetic core coated with a styrene based polymer that is hypercrosslinked via diamines (e.g. TMEDA) and further modified whereby the diamine also serves as a sidechain (i.e. in these types of beads, TMEDA offers both quaternary and tertiary amine functionalities). For a full description of such beads see: WO 2019/141779

In embodiments, the addition of the magnetic beads comprises agitation or mixing. A pre-defined incubation period for capturing the antibiotic analyte(s) of interest on the bead follows. In embodiments, the workflow comprises a washing step (W1) after incubation with the magnetic beads. Depending on the antibiotic analyte(s) one or more additional washing steps (W2) are performed. One washing step (W1, W2 comprises a series of steps including magnetic bead separation by a magnetic bead handling unit comprising magnets or electromagnets, aspiration of liquid, addition of a washing buffer, resuspension of the magnetic beads, another magnetic bead separation step and another aspiration of the liquid. Moreover, washing steps may differ in terms of type of solvent (water/organic/salt/pH), aside from volume and number or combination of washing cycles. It is well-known to the skilled person how to choose the respective parameters. The last washing step (W1, W2) is followed by the addition of an elution reagent followed by resuspension of the magnetic heads and a pre-defined incubation period for releasing the analyte(s) of interest from the magnetic beads. The bound-free magnetic beads are then separated and the supernatant containing derivatized analyte(s) of interest is captured.

In embodiments, a first enrichment workflow comprises addition of magnetic heads carrying matrix-selective groups to the pre-treated sample. In embodiments, the addition of the magnetic beads comprises agitation or mixing. A pre-defined incubation period for capturing the matrix on the bead follows. Here, the analyte of interest does not bind to the magnetic beads but remains in the supernatant. Thereafter, the magnetic beads are separated and the supernatant containing the enriched analyte(s) of interest is collected.

In embodiments, the supernatant is subjected to a second enrichment workflow, in particular to a chromatographic enrichment workflow. In embodiments of the present invention, the chromatographic separation is gas or liquid chromatography. Both methods are well known to the skilled person. In embodiments, the liquid chromatography is selected from the group consisting of HPLC, rapid LC, micro-LC, flow injection, and trap and elute. Here, the supernatant is transferred to the LC station or is transferred to the LC station after a dilution step by addition of a dilution liquid. Different elution procedures/reagents may also be used, by changing e.g. the type of solvents (water/organic/salt/pH) and volume. The various parameters are well-known to the skilled person and easily chosen.

In embodiments, the first enrichment process includes the use of analyte selective magnetic beads. In embodiments, the second enrichment process includes the use of chromatographic separation, in particular using liquid chromatography. In embodiments, the first enrichment process using analyte selective magnetic beads is performed prior to the second enrichment process using liquid chromatography.

In embodiments, determining the amount or concentration of the one or more derivatized antibiotic analyte in the sample, is performed in step b). Any suitable method known to the skilled person may be used. In particular embodiments, step b) comprises determining the amount or concentration of the one or more derivatized antibiotic analyte using immunological methods or mass spectrometry.

In embodiments, wherein step b) comprises determining the amount or concentration of the one or more antibiotic analyte using immunological methods, the following steps are comprised:
i) incubating the (optionally enriched) sample of the patient with one or more antibodies specifically binding to the one or more derivatized antibiotic analyte, thereby generating a complex between the antibody and the one or more derivatized antibiotic analyte, and
ii) quantifying the complex formed in step i), thereby quantifying the amount of the one or more derivatized antibiotic analyte in the sample of the patient.

In particular embodiments, in step i) the sample is incubated with two antibodies, specifically binding to the one or more derivatized antibiotic analyte. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first antibody/derivatized antibiotic analyte/second antibody complex. As the skilled artisan will readily appreciate it is nothing but routine experimentation to establish the time and conditions that are appropriate or that are sufficient for the formation of a complex either between the specific antibody and the derivatized antibiotic analyte or the formation of the secondary, or sandwich complex comprising the first antibody, the derivatized antibiotic analyte, the second antibody.

The detection of the antibody-analyte complex can be performed by any appropriate means. The person skilled in the art is absolutely familiar with such means/methods. In embodiments, the antibody/the antibodies is/are directly or indirectly detectably labeled. In particular embodiments, the antibody is detectably labeled with a luminescent dye, in particular a chemiluminescent dye or an electrochemiluminescent dye.

In embodiments, wherein step b) comprises determining the amount or concentration of the one or more antibiotic derivatized antibiotic analyte using mass spectrometry, the following steps are comprised:

(i) subjecting an ion of the derivatized antibiotic analyte to a first stage of mass spectrometric analysis, whereby the parent ion of the derivatized antibiotic analyte is characterised according to its mass/charge (m/z) ratio,

(ii) causing fragmentation of the derivatized antibiotic analyte parent ion, whereby a daughter ion is generated, wherein the daughter ion of the derivatized antibiotic analyte differs in its m/z ratio from the derivatized antibiotic analyte parent ion, and

(iii) subjecting the daughter ion of the derivatized antibiotic analyte to a second stage of mass spectrometric analysis, whereby the daughter ion of the derivatized antibiotic analyte is characterized according to its m/z ratio.

In embodiments, the parent and/or fragment ions measured are those as indicated in Table 1.

TABLE 1
MRM transitions of Meropenem and Piperacillin:

| | Parent ion (Q1 Mass) | Fragment ion (Q3 Mass) |
|---|---|---|
| Analyte | (Da) | (Da) |
| Meropenem- | 457.164 | 413.2 |
| butylamide | | |
| Meropenem- | 457.164 | 195.2 |
| butylamide | | |
| Meropenem- | 457.164 | 175.1 |
| butylamide | | |
| Piperacillin- | 664.235 | 375.1 |
| (butylamide)₂ | | |
| Piperacillin- | 664.235 | 505.2 |
| (butylamide)₂ | | |
| Piperacillin- | 664.235 | 106.1 |
| (butylamide)₂ | | |

In embodiments, the parent ion of derivatized Meropenem+H⁺ is measured at a m/z value 457.164±0.5, and the parent ion of derivatized Piperacillin+H⁺ is measured at a m/z value 664.235±0.5.

In embodiments, the fragment ion of derivatized Meropenem is measured at a m/z value 152±0.5 or 173±0.5, and the fragment ion of derivatized Piperacillin is measured at a m/z value 270±0.5 or 464±0.5.

In embodiments, the method is an automated method. In particular embodiments, the method is performed by an automated system. In particular embodiments, the method comprises no manual intervention.

In a second aspect, the present invention relates to a method of determining the amount or concentration of one or more antibiotic analytes in an obtained sample, comprising
a) pre-treating the sample with a derivatization reagent, wherein the derivatization reagent comprises a nucleophile,
b) optionally enriching the sample obtained after step a), in particular using magnet beads, and
c) determining the amount or concentration of the one or more antibiotic analyte in the pre-treated sample obtained after step a) or after the optional enrichment step b).

In embodiments, the antibiotic analyte is a lactam antibiotic analyte. In embodiments, the antibiotic analyte is a β-lactam antibiotic analyte. In particular embodiments, the antibiotic analytes is selected from the group consisting of Amoxicillin, Ampicillin, Bacampicillin, Carbenicillin, Cloxacillin, Dicloxacillin, Flucloxillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.

In embodiments, in step a) the sample is pre-treated with a nucleophilic derivatization reagent comprising an amine group, in particular a primary or secondary amine, in particular a primary amine group. In embodiments, in step a) the sample is pre-treated with a nucleophilic derivatization reagent comprises more than 3 C-atoms, in particular 3 to 20 C-atoms, in particular 3 to 10 C-atoms, in particular 3-5 C-atoms, in particular 4 C-atoms. In embodiments in step a) the sample is pre-treated with a linear or branched nucleophilic derivatization reagent, in particular with a linear amine, in particular with a linear primary amine, in particular with a linear primary amine comprising 3 to 5 C-atoms. In embodiments, in step a) the sample is pre-treated with a nucleophilic derivatization reagent selected from the group consisting of propylamine, butylamine, or pentylamine, in particular primary linear butylamine.

In particular embodiments, in step a) the sample is pre-treated with a nucleophilic derivatization reagent comprising butylamine in case the analyte is Meropenem.

In particular embodiments, in step a) the sample is pre-treated with a nucleophilic derivatization reagent comprising pentylamine in case the analyte is Piperacillin.

In embodiments, in step a) the sample is pre-treated with a nucleophilic derivatization reagent comprised in solvent, in particular a solvent selected from the group consisting of water, CH₃CN, THF, Dioxanes, DMF, DMSO, acetone, t-butyl alcohol, diglyme, DME, MeOH, EtOH, 1-PrOH, 2-PrOH, ethylene glycol, Hexamethylphosphoramiede (HMPA), Hexamethylphosphorous triamide (HMPT), and glycerin, in particular a solvent selected from the group consisting of water, CH₃CN, THF, Dioxanes, DMF, DMSO, acetone, t-butyl alcohol, diglyme, and DME.

In embodiments, in step a) the sample is pre-treated with a nucleophilic derivatization reagent comprised in solvent further comprising a non-nucleophilic base that is stable and miscible with water, in particular selected from the group consisting of DBU, TEA, DIPEA, Na₃PO₄, Na₂CO₃, and Cs₂CO₃

In embodiments, in step a) the sample is pre-treated with a nucleophilic derivatization reagent immediately after the sample is obtained, in particular within less than 10 min after the sample was obtained, in particular within less than 5 min after the sample was obtained.

In embodiments, in step a) the sample is pre-treated with a nucleophilic derivatization reagent sample for more than 2 min, in particular more than 5 min, in particular more than 30 min.

In embodiments, the sample obtained after step a) comprises derivatized antibiotic analytes, in particular antibiotic analytes derivatized with a nucleophilic derivatization reagent.

In embodiments, the sample obtained after step a) comprises derivatized β-lactam antibiotic analytes, wherein the beta-lactam moiety is disrupted by the reaction with the nucleophile derivatization reagent. In embodiments, the sample obtained after step a) comprises derivatized β-lactam antibiotic analytes, wherein a covalent adduct of the antibiotic analyte and the nucleophilic derivatization reagent is formed.

In embodiments, the sample obtained after step a) comprises derivatized antibiotic analyte, which is derivatized in at least one of its chemical moieties. The person skilled in the art of chemistry is well aware of chemical moieties which are suitable to be derivatized, in particular with a nucleophilic derivatization reagent. In particular embodiments, the sample obtained after step a) comprises derivatized antibiotic analyte which is derivatized in one, two or three of its chemical moieties.

In particular embodiments, wherein the antibiotic analyte is Meropenem, the sample obtained after step a) comprises derivatized Meropenem, in particular Meropenem derivatized with a nucleophilic derivatization reagent comprising butylamine. See also FIG. 3.

In particular embodiments, wherein the antibiotic analyte is Piperacillin, the sample obtained after step a) comprises derivatized Piperacillin, in particular Piperacillin derivatized with a nucleophilic derivatization reagent comprising butylamine or pentyamine. See also FIG. 4.

In particular embodiments, wherein the antibiotic analyte is Piperacillin, the sample obtained after step a) comprises derivatized Piperacillin, in particular Piperacillin derivatized with a nucleophilic derivatization reagent comprising butylamine or pentyamine, at two of its chemical moieties, in particular derivatized at the β-lactam ring and at the piperazine ring. See also FIG. 4.

In embodiments, additional pre-treatment methods may be performed in step a). These may be performed before or after pre-treating the sample with a derivatization reagent. The pre-treatment method is dependent upon the type of sample, such as blood (fresh or dried), plasma, serum, urine, or saliva, whereas the enrichment method is dependent on the analyte of interest. It is well known to the skilled person which pre-treatment method is suitable for which sample type. It is also well-known to the skilled person which enrichment method is suitable for which analyte of interest.

In embodiments, wherein the sample is a whole blood sample, it is assigned to one of two pre-defined sample pre-treatment (PT) workflows, both comprising the addition of an internal standard (ISTD) and a hemolysis reagent (HR) followed by a pre-defined incubation period (Inc), where the difference between the two workflows is the order in which the internal standard (ISTD) and a hemolysis reagent (HR) are added. In embodiments, the ISTD is added first to the obtained sample followed by the addition of the hemolysis reagent. In embodiments, the ISTD is added to the obtained sample subsequent to the addition of the hemolysis reagents. In embodiments water is added as a hemolysis reagents, in particular in an amount of 0.5:1 to 20:1 mL water/mL sample, in particular in an amount of 1:1 to 10:1 mL water/mL sample, in particular in an amount of 2:1 to 5:1 mL water/mL sample.

In embodiments, wherein the sample is a urine sample, it is assigned to one of other two pre-defined sample PT workflows, both comprising the addition of an ISTD and an enzymatic reagent followed by a pre-defined incubation period, where the difference between the two workflows is the order in which the internal standard and an enzymatic reagent are added. In embodiments, the ISTD is added first to the obtained sample followed by the addition of the enzymatic reagent. In embodiments, the ISTD is added to the obtained sample subsequent to the addition of the enzymatic reagents. An enzymatic reagent is typically a reagent used for glucuronide cleavage or protein cleavage or any pre-processing of analyte or matrix. In embodiments, the enzymatic reagent in selected from the group consisting of glucuronidase, (partial) exo- or endo-deglycoslation enzymes, or exo- or endo proteases. In embodiments, glucuronidase is added in amount of 0.5-10 mg/ml, in particular in an amount of 1 to 8 mg/ml, in particular in an amount of 2 to 5 mg/ml.

In embodiments, wherein the sample is plasma or serum it is assigned to another pre-defined PT workflow including only the addition of an internal standard (ISTD) followed by a pre-defined incubation time.

It is well-known to the skilled person which incubation time and temperature to choose for a sample treatment, chemical reaction or method step considered and as named herein above or below. In particular, the skilled person knows that incubation time and temperature depend upon each other, in that e.g. a high temperature typically leads to a shorter incubation period and vice versa.

The pre-treated sample may be further subjected to at least one enrichment workflow in step b). The enrichment workflow may include one or more enrichment methods. Enrichment methods are well-known in the art and include but are not limited to chemical enrichment methods including but not limited to chemical precipitation, and enrichment methods using solid phases including but not limited to solid phase extraction methods, bead workflows, and chromatographic methods (e.g. gas or liquid chromatography).

In embodiments, a first enrichment workflow comprises the addition of a solid phase, in particular of solid beads, carrying analyte-selective groups to the pre-treated sample.

In embodiments, a first enrichment workflow, comprises the addition of magnetic or paramagnetic beads carrying analyte-selective groups to the pre-treated sample. In embodiments, the magnetic beads comprise a magnetic core coated with a styrene based polymer that is hypercrosslinked via Friedel-Crafts alkylation and further modified with addition of —OH groups. In embodiments, the magnetic beads comprise a magnetic core coated with a styrene based polymer that is hypercrosslinked via diamines (e.g. tetramethylendiamine (TMEDA)) and further modified whereby the diamine also serves as a sidechain (i.e. Diamine Beads with TMEDA offer both quaternary and tertiary amine functionalities). For a full description see e.g. WO 2019/141779.

In embodiments, the enrichment workflow in step b) using magnetic beads comprises agitation or mixing. A pre-defined incubation period for capturing the antibiotic analyte(s) of interest on the bead follows. In embodiments, the workflow comprises a washing step (W1) after incubation with the magnetic beads. Depending on the antibiotic analyte(s) one or more additional washing steps (W2) are performed. One washing step (W1, W2) comprises a series of steps including magnetic head separation by a magnetic bead handling unit comprising magnets or electromagnets, aspiration of liquid, addition of a washing buffer, resuspension of the magnetic beads, another magnetic bead separation step and another aspiration of the liquid. Moreover, washing steps may differ in terms of type of solvent (water/organic/salt/pH), apart from volume and number or combination of washing cycles. It is well-known to the skilled person how to choose the respective parameters. The last washing step (W1, W2) is followed by the addition of an elution reagent followed by resuspension of the magnetic beads and a pre-defined incubation period for releasing the analyte(s) of interest from the magnetic beads. The bound-free magnetic beads are then separated and the supernatant containing derivatized analyte(s) of interest is captured.

In embodiments, a first enrichment workflow comprises the addition of magnetic beads carrying matrix-selective groups to the pre-treated sample. In embodiments, the addition of the magnetic beads comprises agitation or mixing. A pre-defined incubation period for capturing the matrix on the bead follows. Here, the analyte of interest does not bind to the magnetic beads but remains in the supernatant. Thereafter, the magnetic beads are separated and the supernatant containing the enriched analyte(s) of interest is collected. In embodiments, the supernatant is subjected to a second enrichment workflow, in particular to a chromatographic enrichment workflow. In embodiments, the chromatographic separation is gas or liquid chromatography. Both methods are well known to the skilled person. In embodiments, the liquid chromatography is selected from the group consisting of HPLC, rapid LC, micro-LC, flow injection, and trap and elute. Here, the supernatant is transferred to the LC station or is transferred to the LC station after a dilution step by addition of a dilution liquid. Different elution procedures/reagents may also be used, by changing e.g. the type of solvents (water/organic/salt/pH) and volume. The various parameters are well-known to the skilled person and easily chosen.

In particular embodiments, the first enrichment process includes the use of analyte selective magnetic beads. In embodiments, the second enrichment process includes the use of chromatographic separation, in particular using liquid chromatography. In embodiments, the first enrichment process using analyte selective magnetic beads is performed prior to the second enrichment process using liquid chromatography.

In embodiments determining the amount or concentration of the one or more derivatized antibiotic analyte in the sample, is performed in step c). Any suitable method known to the skilled person may be used. In particular embodiments, step c) comprises determining the amount or concentration of the one or more derivatized antibiotic analyte using immunological methods or mass spectrometry.

In embodiments, wherein step c) comprises determining the amount or concentration of the one or more antibiotic analyte using immunological methods, the following steps are comprised:
i) incubating the sample of the patient with one or more antibodies specifically binding to the one or more derivatized antibiotic analyte, thereby generating a complex between the antibody and the one or more derivatized antibiotic analyte, and
ii) quantifying the complex formed in step if thereby quantifying the amount of the one or more antibiotic analyte in the sample of the patient.

In particular embodiments, in step i) the sample is incubated with two antibodies, specifically binding to the one or more derivatized antibiotic analyte. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first antibody/derivatized antibiotic analyte/second antibody complex. As the skilled artisan will readily appreciate it is nothing but routine experimentation to establish the time and conditions that are appropriate or that are sufficient for the formation of a complex either between the specific antibody and the derivatized antibiotic analyte or the formation of the secondary, or sandwich complex comprising the first antibody, the derivatized antibiotic analyte, the second antibody.

The detection of the antibody-analyte complex can be performed by any appropriate means. The person skilled in the art is absolutely familiar with such means/methods. In embodiments, the antibody/the antibodies is/are directly or indirectly detectably labeled. In particular embodiments, the antibody is detectably labeled with a luminescent dye, in particular a chemiluminescent dye or an electrochemiluminescent dye.

In embodiments, wherein step c) comprises determining the amount or concentration of the one or more antibiotic derivatized antibiotic analyte using mass spectrometry, the following steps are comprised:

(i) subjecting an ion of the derivatized antibiotic analyte to a first stage of mass spectrometric analysis, whereby the parent ion of the derivatized antibiotic analyte is characterised according to its mass/charge (m/z) ratio,

(ii) causing fragmentation of the derivatized antibiotic analyte parent ion, whereby a daughter ion is generated, wherein the daughter ion of the derivatized antibiotic analyte differs in its m/z ratio from the derivatized antibiotic analyte parent ion, and

(iii) subjecting the daughter ion of the derivatized antibiotic analyte to a second stage of mass spectrometric analysis, whereby the daughter ion of the derivatized antibiotic analyte is characterized according to its m/z ratio.

In embodiments, the parent and/or fragment ions measured are those as indicated in Table 1.

In embodiments, the parent ion of derivatized Meropenem+H⁺ is measured at an m/z value 457.164±0.5, and the parent ion of derivatized Piperacillin+H⁺ is measured at an m/z value 664.235±0.5.

In embodiments, the fragment ion of derivatized Meropenem is measured at an m/z value 152±0.5 or 173±0.5, and the fragment ion of derivatized Piperacillin is measured at an m/z value 270±0.5 or 464±0.5.

In embodiments, the method is an automated method. In particular embodiments, the method is performed by an automated system. In particular embodiments, the method comprises no manual intervention.

In a third aspect, the present invention relates to an analytical system adapted to perform the method of the first or the second aspect.

In embodiments, the system is a mass spectrometry system, in particular an LC/MS system. In embodiments, the analytical system is an automated analytical system. In particular embodiments, the analytical system does not require manual intervention, i.e. the operation of the system is purely automated. In particular embodiments, the LC/MS system is an automated, random-access LC/MS system. In embodiments, the MS device is a tandem mass spectrometer, in particular a triple quadrupole device. In embodiments, the LC is HPLC, in particular is RP-HPLC, or rapid LC. In embodiments, the ion formation is based on electrospray ionization (ESI) or atmospheric pressure chemical ionization (APCI), in particular positive polarity mode ESI.

In a fourth aspect, the present invention relates to a sampling tube for collecting a patient sample comprising a nucleophilic derivatization reagent suitable to stabilize one or more antibiotic analytes in a sample. In embodiments, the present invention relates to a sampling tube for collecting a patient sample comprising a nucleophilic derivatization reagent which stabilizes one or more antibiotic analytes in a sample.

Sample collections tubes suitable to be used for collecting a patient sample are well-known in the art and are used on a routine basis by practioners. As the skilled artisan will appreciate the sampling tube preferably will in fact be a tube. In particular, the sampling tube has a size and dimension adapted to match the requirements of the sample receiving station of an automated analyzer, e.g. an Elecsys® analyzer of Roche Diagnostics. The sampling tube may have a conical or preferably a round bottom. In clinical routine standard tube sizes are used that are compatible with the analyzers systems on the market. Standard and preferred tubes e.g. have the following dimensions: 13×75 mm; 13×100 mm, 16×100 mm.

In embodiments, the sampling tube according to the present invention is only used once, i.e, it is a single use device. In particular embodiments, the sampling tube according to the present invention is not only appropriate for collection of a sample but it is also adapted to allow for the further processing of the sample. By collecting a sample into a sampling tube containing the nucleophilic derivatization reagent, the desired result, i.e. the derivatization of the antibiotic analyte, is achieved.

In embodiments, the nucleophilic derivatization reagent comprises an amine group, in particular a primary or secondary amine, in particular a primary amine group. In embodiments, the nucleophilic derivatization reagent comprises more than 3 C-atoms, in particular 3 to 20 C-atoms, in particular 3 to 10 C-atoms, in particular 3-5 C-atoms, in particular 4 C-atoms. In embodiments, the nucleophilic derivatization reagent is linear or branched, in particular with a linear amine, in particular with a linear primary amine, in particular with a linear primary amine comprising 3 to 5 C atoms. In embodiments, the nucleophilic derivatization reagent is selected from the group consisting of propylamine, butylamine, or pentylamine, in particular primary linear butylamine or primary linear pentylamine.

In embodiments, the nucleophilic derivatization reagent derivatizes the antibiotic analyte in at least one of its chemical moieties. The person skilled in the art of chemistry is well-aware of chemical moieties which are suitable to be derivatized, in particular with a nucleophilic derivatization reagent. In particular embodiments, the nucleophilic derivatization reagent derivatizes antibiotic analyte in one, two or three of its chemical moieties.

In particular embodiments, the nucleophilic derivatization reagent comprises butylamine in case the antibiotic analyte is Meropenem.

In particular embodiments, the nucleophilic derivatization reagent comprises pentylamine in case the antibiotic analyte is Piperacillin.

In embodiments, the nucleophilic derivatization reagent is comprised in liquid or lyophilized form. In embodiments, the nucleophilic derivatization reagent further comprises a non-nucleophilic base that is stable and miscible with water, in particular selected from the group consisting of DBU, TEA, DIPEA, Na₃PO₄, Na₂CO₃, and Cs₂CO₃. In embodiments, the nucleophilic derivatization reagent is comprised in liquid form comprised in a solvent, in particular a solvent selected from the group consisting of water, CH₃CN, THF, Dioxanes, DMF, DMSO, acetone, t-butyl alcohol, diglyme, DME, MeOH, EtOH, 1-PrOH, 2-PrOH, ethylene glycol, Hexamethylphosphoramiede (HMPA), Hexamethylphosphorous triamide (HMPT), and glycerin, in particular a solvent selected from the group consisting of water, CH₃CN, THF, Dioxanes, DMF, DMSO, acetone, tBuOH, diglyme, and DME.

In a fifth aspect, the present invention relates to the use of a nucleophilic derivatization reagent for determining the amount or concentration of one or more antibiotic analytes in a sample.

In embodiments, the nucleophilic derivatization reagent is a reagent comprising an amine group, in particular a primary or secondary amine, in particular a primary amine group. In embodiments, the nucleophilic derivatization reagent comprises more than 3 C-atoms, in particular 3 to 20 C-atoms, in particular 3 to 10 C-atoms, in particular 3-5 C-atoms, in particular 4 C-atoms. In embodiments, the nucleophilic derivatization reagent is linear or branched, in particular a linear amine, in particular a linear primary amine, in particular a linear primary amine comprising 3 to 5 C-atoms. In embodiments, the derivatization reagent is selected from the group consisting of propylamine, butylamine, or pentylamine, in particular primary linear butylamine.

In embodiments, the antibiotic substance is a β-lactam antibiotic substance. In embodiments, the antibiotic substance is selected from the group consisting of Amoxicillin, Ampicillin, Bacampicillin, Carbenicillin, Cloxacillin, Dicloxacillin, Flucloxacillin, Mezlocillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.

In embodiments, the nucleophilic derivatization reagent stabilizes the antibiotic substance. In embodiments, the nucleophilic derivatization reagent prevents the hydrolyzation of the antibiotic substance during determining the amount or concentration of one or more antibiotic analytes in a sample. In embodiments, the nucleophilic derivatization reagent stabilizes the antibiotic substance by forming a covalent adduct of the antibiotic analyte and the nucleophilic derivatization reagent.

In embodiments, the nucleopilic derivatization reagent stabilizes the antibiotic analyte in at least one of its chemical moieties. The person skilled in the art of chemistry is well aware of chemical moieties which are suitable to be derivatized, in particular with a nucleophilic derivatization reagent. In particular embodiments, the nucleophilic derivatization reagent derivatizes antibiotic analyte in one, two or three of its chemical moieties. In particular embodiments, the nucleophilic derivatization reagent stabilizes the antibiotic analyte by reacting with its β-lactam ring.

In particular embodiments, wherein the antibiotic analyte is Meropenem, a nucleophilic derivatization reagent comprising butylamine is used to stabilize Meropenem. See also FIG. 3

In particular embodiments, wherein the antibiotic analyte is Piperacillin, a nucleophilic derivatization reagent comprising butylamine or pentylamine is used to stabilize Piperacillin. See also FIG. 4

In particular embodiments, wherein the antibiotic analyte is Piperacilin, a nucleophilic derivatization reagent comprising butylamine or pentylamine is used to stabilize Piperacillin at two of its chemical moieties, in particular derivatized at the β-lactam ring and at the piperazine ring. See also FIG. 4

In embodiments, the nucleophilic derivatization reagent stabilized the antibiotic substance for more than 2 hours, for more than 4 hours, for more than 8 hours, for more than 12 hours, for more than 15 hours, for more than 24 hours, for more than 48 hours, for more than 7 days, for more than 2 weeks, for more than 4 weeks, for more than 2 months, for more than 3 months, for more than 4 months, for more than 5 months, or for more than 6 months. In particular embodiments, the nucleophilic derivatization reagent stabilized the antibiotic substance for more than 8 hours, in particular for more than 12 hours. In particular embodiments, the nucleophilic derivatization reagent stabilized the antibiotic substance for more than 15 hours. In particular embodiments, the nucleophilic derivatization reagent stabilized the antibiotic substance for at least 16 hours. In particular embodiments, the nucleophilic derivatization reagent stabilized the antibiotic substance for 16 hours.

In a sixth aspect, the present inventions relates to the use of a nucleophilic derivatization reagent to stabilize an antibiotic analyte in a sample of interest.

In embodiments, the nucleophilic derivatization reagent is a reagent comprising an amine group, in particular a primary or secondary amine, in particular a primary amine group, in embodiments, the nucleophilic derivatization reagent comprises more than 3 C-atoms, in particular 3 to 20 C-atoms, in particular 3 to 10 C-atoms, in particular 3-5 C-atoms, in particular 4 C-atoms. In embodiments, the nucleophilic derivatization reagent is linear or branched, in particular a linear amine, in particular a linear primary amine, in particular a linear primary amine comprising 3 to 5 C-atoms. In embodiments, the derivatization reagent is selected from the group consisting of propylamine, butylamine, or pentylamine, in particular primary linear butylamine.

In embodiments, the antibiotic substance is a β-lactam antibiotic substance. In embodiments, the antibiotic substance is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte Meropenem or Piperacillin.

In embodiments, the nucleophilic derivatization reagent stabilizes the antibiotic substance. In embodiments, the nucleophilic derivatization reagent prevents the hydrolyzation of the antibiotic substance during determining the amount or concentration of one or more antibiotic analytes in a sample. In embodiments, the nucleophilic derivatization reagent stabilizes the antibiotic substance by forming a covalent adduct of the antibiotic analyte and the nucleophile derivatization reagent. In embodiments, the nucleophilic derivatization reagent stabilized the antibiotic substance for more than 2 hours, for more than 4 hours, for more than 8 hours, for more than 12 hours, for more than 15 hours, for more than 24 hours, for more than 48 hours, for more than 7 days, for more than 2 weeks, for more than 4 weeks, for more than 2 months, for more than 3 months, for more than 4 months, for more than 5 months, or for more than 6 months. In particular embodiments, the nucleophilic derivatization reagent stabilized the antibiotic substance for more than 8 hours, in particular for more than 12 hours. In particular embodiments, the nucleophilic derivatization reagent stabilized the antibiotic substance for more than 15 hours. In particular embodiments, the nucleophilic derivatization reagent stabilized the antibiotic substance for at least 16 hours. In particular embodiments, the nucleophilic derivatization reagent stabilized the antibiotic substance for 16 hours.

In a seventh aspect, the present invention relates to an antibiotic analyte stabilized by nucleophilic derivatization reagent.

In embodiments, the nucleophilic derivatization reagent prevents the hydrolyzation of the antibiotic substance during determining the amount or concentration of one or more antibiotic analytes in a sample. In embodiments, the antibiotic substance is stabilized by the nucleophilic derivatization reagent due to the formation of a covalent adduct of the antibiotic analyte and the nucleophilic derivatization reagent. In embodiments, the antibiotic substance is stabilized by the nucleophilic derivatization reagent for more than 2 hours, for more than 4 hours, for more than 8 hours, for more than 12 hours, for more than 15 hours, for more than 24 hours, for more than 48 hours, for more than 7 days, for more than 2 weeks, for more than 4 weeks, for more than 2 months, for more than 3 months, for more than 4 months, for more than 5 months, or for more than 6 months. In particular embodiments, the antibiotic substance is stabilized by the nucleophilic derivatization reagent for more than 8 hours, in particular for more than 12 hours. In particular embodiments, the antibiotic substance is stabilized by the nucleophilic derivatization reagent for more than 15 hours, in particular embodiments, the antibiotic substance is stabilized by the nucleophilic derivatization reagent for at least 16 hours. In particular embodiments, the antibiotic substance is stabilized by the nucleophilic derivatization reagent for 16 hours.

In embodiments, the antibiotic substance is a β-lactam antibiotic substance. In embodiments, the antibiotic substance is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.

In embodiments, the nucleophilic derivatization reagent is a reagent comprising an amine group, in particular a primary or secondary amine, in particular a primary amine group. In embodiments, the nucleophilic derivatization reagent comprises more than 3 C-atoms, in particular 3 to 20 C-atoms, in particular 3 to 10 C-atoms, in particular 3-5 C-atoms, in particular 4 C-atoms. In embodiments, the nucleophilic derivatization reagent is linear or branched, in particular a linear amine, in particular a linear primary amine, in particular a linear primary amine comprising 3 to 5 C-atoms. In embodiments, the derivatization reagent is selected from the group consisting of propylamine, butylamine, or pentylamine, in particular primary linear butylamine.

In embodiments, the antibiotic analyte is stabilized by the nucleophilic derivatization reagent in at least one of its chemical moieties. The person skilled in the art of chemistry is well aware of chemical moieties which are suitable to be derivatized, in particular with a nucleophilic derivatization reagent. In particular embodiments, the antibiotic analyte is derivatized by the nucleophilic derivatization reagent in one, two or three of its chemical moieties. In particular embodiments, the antibiotic analyte is stabilized by the nucleophilic derivatization reagent by reacting with its β-lactam ring.

In particular embodiments, wherein the antibiotic analyte is Meropenem, a nucleophilic derivatization reagent comprising butylamine is used to stabilize Meropenem. See also FIG. 3

In particular embodiments, wherein the antibiotic analyte is Piperacillin, a nucleophilic derivatization reagent comprising butylamine or pentylamine is used to stabilize Piperacillin. See also FIG. 4

In particular embodiments, wherein the antibiotic analyte is Piperacilin, a nucleophilic derivatization reagent comprising butylamine or pentylamine is used to stabilize Piperacillin at two of its chemical moieties, in particular derivatized at the β-lactam ring and at the piperazine ring. See also FIG. 4

The present invention further relates to the following items:
1) An (automated) method of determining the amount or concentration of one or more derivatized antibiotic analytes in an obtained sample comprising
   a) optionally pre-treating and/or enriching the sample, in particular using magnetic beads, and
   b) determining the amount or concentration of the one or more antibiotic analyte in the sample.
2) The method of item 1, wherein the antibiotic analyte is a lactam antibiotic analyte.
3) The method of item 1 or 2, wherein the antibiotic analyte is a β-lactam antibiotic analyte.
4) The method of any of items 1 to 3, wherein the antibiotic analytes is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.
5) The method of any of items 1 to 4, wherein the antibiotic analyte is Meropenem or piperacillin.
6) The method of any of items 1 to 5, wherein the antibiotic analyte is derivatized with a nucleophilic derivatization reagent, in particular a reagent comprising an amine group, in particular a primary or secondary amine, in particular a primary amine group.
7) The method of any of items 1 to 6, wherein the antibiotic analyte is derivatized with a nucleophilic derivatization reagent comprises more than 3 C-atoms, in particular 3 to 20 C-atoms, in particular 3 to 10 C-atoms, in particular 3-5 C-atoms, in particular 4 C-atoms.
8) The method of any of items 1 to 7, wherein the antibiotic analyte is derivatized with a linear or branched, nucleophilic derivatization reagent, in particular with a liner amine, in particular with a linear primary amine, in particular with a linear primary amine comprising 3 to 5 C-atoms.
9) The method of any of items 1 to 8, wherein the antibiotic analyte is derivatized with a nucleophilic derivatization reagent selected from the group consisting of propylamine, butylamine, or pentylamine, in particular primary linear butylamine.
10) The method of any of items 1 to 9, wherein enrichment step a) comprises at least one enrichment workflow,
11) The method of any of items 1 to 9, wherein enrichment step a) comprises using magnetic beads, in particular type A or B magnetic beads.
12) The method of any of items 1 to 11, wherein enrichment step a) comprises two enrichments steps, in particular a first enrichment step comprising using magnetic beads, and a second enrichment step using evaporation.
13) The method of any of items 1 to 12, wherein in step b) the amount or concentration of the derivatized antibiotic analyte is determined using immunological assay or LC/MS
14) The method of any of items 1 to 13, wherein in step b) the amount or concentration of the derivatized antibiotic analyte is determined using LC/MS, wherein the LC is HPLC, in particular is RP-HPLC, or rapid LC.
15) The method of any of items 1 to 14, wherein in step b) the amount or concentration of the derivatized antibiotic analyte is determined using LC/MS, wherein the ion formation is based on electrospray ionization (ESI), in particular positive polarity mode ESI.
16) The method of any of items 1 to 15, wherein in step b) the amount or concentration of the derivatized antibiotic analyte is determined using LC/MS, wherein the MS device is a tandem mass spectrometer, in particular a triple quadrupole device, in particular an automated, random-access LC/MS system.
17) The method of any of items 1 to 16, wherein in step b) the amount or concentration of the derivatized antibiotic analyte is determined using LC/MS, wherein the parent ion of derivatized Meropenem+H⁺ is measured at an m/z value 457.164±0.5, and the parent ion of derivatized Piperacillin+H⁺ is measured at an m/z value 664.235±0.5.
18) The method of any of items 1 to 17, wherein in step b) the amount or concentration of the derivatized antibiotic analyte is determined using LC/MS, wherein the fragment ion of derivatized Meropenem is measured at an m/z value 152±0.5 or 173±0.5, and the fragment ion of derivatized Piperacillin is measured at an m/z value 270±0.5 or 464±0.5.
19) An (automated) method of determining the amount or concentration of one or more antibiotic analytes in an obtained sample, comprising
   a) pre-treating the sample with a derivatization reagent, wherein the derivatization reagent comprises a nucleophile,
   b) optionally enriching the sample obtained after step a), in particular using magnetic beads, and
   c) determining the amount or concentration of the one or more antibiotic analyte(s) in the pre-treated sample obtained after step a) or after the optional enrichment step b).
20) The method of item 19, wherein the antibiotic analyte is a lactam antibiotic analyte.
21) The method of item 19 or 20, wherein the antibiotic analyte is a β-lactam antibiotic analyte.
22) The method of any of items 19 to 21, wherein the antibiotic analytes is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.
23) The method of any of items 19 to 22, wherein the antibiotic analyte is Meropenem or Piperacillin.
24) The method of any of items 19 to 23, wherein in step a) the sample is pre-treated with a nucleophilic derivatization reagent comprising an amine group, in particular a primary or secondary amine, in particular a primary amine group.
25) The method of any of items 19 to 24, wherein in step a) the sample is pre-treated with a nucleophilic derivatization reagent comprises more than 3 C-atoms, in particular 3 to 20 C-atoms, in particular 3 to 10 C-atoms, in particular 3-5 C-atoms, in particular 4 C-atoms.
26) The method of any of items 19 to 25, wherein in step a) the sample is pre-treated with a linear or branched nucleophilic derivatization reagent, in particular with a linear amine, in particular with a linear primary amine, in particular with a linear primary amine comprising 3 to 5 C-atoms.
27) The method of any of items 19 to 28, wherein in step a) the sample is pre-treated with a nucleophilic derivatization reagent selected from the group consisting of propylamine, butylamine, or pentylamine, in particular primary linear butylamine.
28) The method of any of items 19 to 27, wherein in step a) the sample is pre-treated with a nucleophilic derivatization reagent comprised in solvent, in particular a solvent selected from the group consisting of water, CH₃CN, THF, Dioxanes, DMF, DMSO, acetone, t-butyl alcohol, diglyme, DME, MeOH, EtOH, 1-PrOH, 2-PrOH, ethylene glycol, Hexamethylphosphoramiede (HMPA), Hexamethylphosphorous triamide (HMPT), and glycerin, in particular a solvent selected from the group consisting of water, CH₃CN, THF, Dioxanes, DMF, DMSO, acetone, tBuOH, diglyme, and DME.
29) The method of any of items 19 to 28, wherein in step a) the sample is pre-treated with a nucleophilic derivatization reagent comprised in solvent further comprising a non-nucleophilic base that is stable and miscible with water, in particular selected from the group consisting of DBU, TEA, DIPEA, Na₃PO₄, Na₂CO₃, and Cs₂CO₃
30) The method of any of items 19 to 29, wherein in step a) the sample is pre-treated with a nucleophilic derivatization reagent comprising butylamine in case the analyte is Meropenem.
31) The method of any of items 19 to 30, wherein in step a) the sample is pre-treated with a nucleophilic derivatization reagent comprising pentylamine in case the analyte is Piperacillin.
32) The method of any of items 19 to 31, wherein in step a) the sample is pre-treated with a nucleophilic derivatization reagent immediately after the sample is obtained, in particular within less than 10 min after the sample was obtained, in particular within less than 5 min after the sample was obtained.
33) The method of any of items 19 to 31, wherein in step a) the sample is pre-treated with a nucleophilic derivatization reagent sample for more than 2 min, in particular more than 5 min, in particular more than 30 min.
34) The method of any of items 19 to 33, wherein the sample obtained after step a) comprises derivatized antibiotic analytes, in particular antibiotic analytes derivatized with a nucleophilic derivatization reagent.
35) The method of any of items 19 to 34, wherein the sample obtained after step a) comprises derivatized β-lactam antibiotic analytes, wherein the beta-lactam moiety is disrupted by the reaction with the nucleophilic derivatization reagent.
36) The method of any of items 19 to 35, wherein enrichment step b) comprises at least one enrichment workflow.
37) The method of any of items 19 to 36, wherein enrichment step b) comprises using magnetic beads, in particular type A or B magnetic beads.
38) The method of any of items 19 to 37, wherein enrichment step b) comprises two enrichments steps, in particular a first enrichment step comprising magnetic beads, and a second enrichment step using evaporation.
39) The method of any of items 19 to 38, wherein in step c) the amount or concentration of the antibiotic analyte is determined using immunological assay or LC/MS
40) The method of any of items 19 to 39, wherein in step c) the amount or concentration of the antibiotic analyte is determined using LC/MS, wherein the LC is HPLC, in particular is RP-HPLC, or rapid LC.
41) The method of any of items 19 to 40, wherein in step c) the amount or concentration of the antibiotic analyte is determined using LC/MS, wherein the ion formation is based on electrospray ionization (ESI), in particular positive polarity mode ESI.
42) The method of any of items 19 to 41, wherein in step c) the amount or concentration of the antibiotic analyte is determined using LC/MS, wherein the MS device is a tandem mass spectrometer, in particular a triple quadrupole device, in particular an automated, random-access LC/MS system.
43) The method of any of items 19 to 42, wherein in step c) the amount or concentration of the antibiotic analyte is determined using LC/MS, wherein the parent ion of derivatized Meropenem+H⁺ is measured at a m/z value 457.164±0.5, and the parent ion of derivatized Piperacillin+H⁺ is measured at a m/z value 664.235±0.5.
44) The method of any of items 19 to 43, wherein in step c) the amount or concentration of the antibiotic analyte is determined using LC/MS, wherein the fragment ion of derivatized Meropenem is measured at a m/z value 152±0.5 or 173±0.5, and the fragment ion of derivatized Piperacillin is measured at a m/z value 270+0.5 or 464+0.5.
45) An (automated) analytical system (in particular LC/MS system) adapted to perform the method of any of items 1 to 44.
46) A sampling tube for collecting a patient sample comprising a nucleophilic derivatization reagent suitable to stabilize one or more antibiotic analytes in a sample.
47) A sampling tube for collecting a patient sample comprising: a device with a reservoir adapted for receiving a blood sample to be collected, and a nucleophilic derivatization reagent suitable to stabilize one or more antibiotic analytes in a sample.
48) The sampling tube of item 46 or 47, wherein the nucleophilic derivatization reagent comprising an amine group, in particular a primary or secondary amine, in particular a primary amine group.
49) The sampling tube of any of items 46 to 48, wherein the nucleophilic derivatization reagent comprises more than 3 C-atoms, in particular 3 to 20 C-atoms, in particular 3 to 10 C-atoms, in particular 3-5 C-atoms, in particular 4 C-atoms.
50) The sampling tube of any of items 46 to 49, wherein the nucleophilic derivatization reagent is linear or branched, in particular with a linear amine, in particular with a linear primary amine, in particular with a linear primary amine comprising 3 to 5 C-atoms.
51) The sampling tube of any of items 46 to 50, wherein the nucleophilic derivatization reagent is selected from the group consisting of propylamine, butylamine, or pentylamine, in particular primary linear butylamine.
52) The sampling tube of any of items 46 to 51, wherein the nucleophilic derivatization reagent is comprised in liquid or lyophilized form.
53) The sampling tube of any of items 46 to 52, wherein the nucleophilic derivatization reagent further comprises a non-nucleophilic base that is stable and miscibile with water, in particular selected from the group consisting of DBU, TEA, DIPEA, Na₃PO₄, Na₂CO₃, and Cs₂CO₃.
54) The sampling tube of any of items 46 to 53, wherein the nucleophilic derivatization reagent is comprised in liquid form comprised in a solvent, in particular a solvent selected from the group consisting of water, CH₃CN, THF, Dioxanes, DMF, DMSO, acetone, tBuOH, diglyme, DME, MeOH, EtOH-1-PrOH, 2-PrOH, ethylene glycol, Hexamethylphosphoramiede (HMPA), Hexamethylphosphorous triamide (HMPT), and glycerin, in particular a solvent selected from the group consisting of water, CH₃CN, THF, Dioxanes, DMF, DMSO, acetone, tBuOH diglyme, and DME.
55) The sampling tube of any of items 46 to 54, wherein the nucleophilic derivatization reagent comprises butyl imine in case the antibiotic analyte is Meropenem.
56) The sampling tube of any of items 46 to 55, wherein the nucleophilic derivatization reagent comprises pentylamine in case the antibiotic analyte is Piperacillin.
57) Use of a nucleophilic derivatization reagent for determining the amount or concentration of one or more antibiotic analytes in a sample.
58) The use of item 57, wherein the nucleophilic derivatization reagent is a reagent comprising an amine group, in particular a primary or secondary amine, in particular a primary amine group.
59) The use of item 57 or 58, wherein the nucleophilic derivatization reagent comprises more than 3 C-atoms, in particular 3 to 20 C-atoms, in particular 3 to 10 C-atoms, in particular 3-5 C-atoms, in particular 4 C-atoms.
60) The use of any of items 57 to 59, wherein the nucleophilic derivatization reagent is linear or branched, in particular a linear amine, in particular a linear primary amine, in particular a linear primary amine comprising 3 to 5 C-atoms.
61) The use of any of items 57 to 60, wherein the derivatization reagent is selected from the group consisting of propylamine, butylamine, or pentylamine, in particular primary linear butylamine.
62) The use of any of items 57 to 61, wherein the antibiotic substance is a β-lactam antibiotic substance.
63) The use of any of items 57 to 62, wherein the antibiotic substance is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.
64) The use of any of items 57 to 63, wherein the antibiotic substance is Meropenem or Piperacillin.
65) The use of any of items 57 to 63, wherein the nucleophilic derivatization reagent prevents the hydrolyzation of the antibiotic substance during determining the amount or concentration of one or more antibiotic analytes in a sample.
66) The use of any of items 57 to 65, wherein the nucleophilic derivatization reagent stabilized the antibiotic substance for more than 7 days, for more than 2 weeks, for more than 3 weeks, for more than 4 weeks, for more than 2 months, for more than 3 months, for more than 4 months, for more than 5 months, or for more than 6 months.
67) Use of a nucleophilic derivatization reagent to stabilize an antibiotic analyte in a sample of interest.
68) The use of item 67, wherein the nucleophilic derivatization reagent is an reagent comprising an amine group, in particular a primary or secondary amine, in particular a primary amine group.
69) The use of item 67 or 68, wherein the nucleophilic derivatization reagent comprises more than 3 C-atoms, in particular) 20 C-atoms, in particular 3 to 10 C-atoms, in particular 3-5 C-atoms, in particular 4 C-atoms.
70) The use of any of items 67 to 69 wherein the nucleophilic derivatization reagent is linear or branched, in particular a linear amine, in particular a linear primary amine, in particular a linear primary amine comprising 3 to 5 C-atoms.
71) The use of any of items 6 to 70, wherein the derivatization reagent is selected from the group consisting of propylamine, butylamine, or pentylamine, particular primary linear butylamine.
72) The use of any of items 67 to 71, wherein the antibiotic substance is a β-lactam antibiotic substance.
73) The use of any of items 67 to 72, wherein the antibiotic substance is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefemipidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.
74) The use of any of items 67 to 73, wherein the antibiotic substance is Meropenem or Piperacillin.
75) The use of any of items 67 to 74, wherein the nucleophilic derivatization reagent prevents the hydrolyzation of the antibiotic substance.
76) The use of any of items 67 to 75, wherein the nucleophilic derivatization reagent stabilized the antibiotic substance for more than 7 days, for more than 2 weeks, for more than 3 weeks, for more than 4 weeks, for more than 2 months, for more than 3 months, for more than 4 months, for more than 5 months, or for more than 6 months.
77) An antibiotic analyte stabilized by nucleophilic derivatization reagent.
78) The antibiotic analyte of item 77, wherein the nucleophilic derivatization reagent is a reagent comprising an amine group, in particular a primary or secondary amine, in particular a primary amine group.
79) The antibiotic analyte of item 77 or 78, wherein the nucleophilic derivatization reagent comprises more than 3 C-atoms, in particular 3 to 20 C-atoms, in particular 3 to 10 C-atoms, in particular 3-5 C-atoms, in particular 4 C-atoms.
80) The antibiotic analyte of any of items 77 to 79, wherein the nucleophilic derivatization reagent is linear or branched, in particular a linear amine, in particular a linear primary amine, in particular a linear primary amine comprising 3 to 5 C-atoms.
81) The antibiotic analyte of any of items 77 to 80, wherein the derivatization reagent is selected from the group consisting of propylamine, butylamine, or pentylamine, in particular primary linear butylamine.
82) The antibiotic analyte of any of items 77 to 81, wherein the antibiotic substance is a β-lactam antibiotic substance.
83) The antibiotic analyte of any of items 77 to 82, wherein the antibiotic substance is selected from the group consisting of Amoxicillin, Ampicillin, Bacampicillin, Carbenicillin, Cloxacillin, Dicloxacillin, Flucloxacillin, Mezlocillin, Nafcillin, Oxacillin, Temocillin, Pheneticillin, Penicillin G, Penicillin V, Piperacillin, Azlocillin, Pivampicillin, Pivmecillinam, Ticarcillin, Cefacetrile (cephacetrile), Cefadroxil (cefadroxyl), Cefalexin (cephalexin), Cefalexin (cephalexin), Cefaloglycin (cephaloglycin), Cefalonium (cephalonium), Cefaloridine (cephaloradine), Cefalotin (cephalothin), Cefalotin (cephalothin), Cefapirin (cephapirin), Cefatrizine, Cefazaflur, Cefazedone, Cefazolin (cephazolin), Cefradine (cephradine), Cefradine (cephradine), Cefroxadine, Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefalcram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Ceftolozane, Imipenem, Doripenem, Ertapenem, Meropenem, Aztreonam, Mecillinam, Metampicillin, Talampicillin, Epicillin, Sulbenicillin, Faropenem, Ritipenem, Biapenem, Pivampicillin, Clometocillin, Penamecillin, Hetacillin, Carindacillin, Panipenem, Tigemonam, Carumonam, Nocardicin A, Penam, Sulbactam, Tazobactam, Clavam, and Clavulanic acid. In particular embodiments, the antibiotic analyte is Meropenem or Piperacillin.
84) The antibiotic analyte of any of items 77 to 83, wherein the antibiotic substance is Meropenem or Piperacillin.
85) The antibiotic analyte of any of items 77 to 83, wherein the nucleophilic derivatization reagent prevents the hydrolyzation of the antibiotic substance during determining the amount or concentration of one or more antibiotic analytes in a sample.
86) The antibiotic analyte of any of items 77 to 85 wherein derivatized β-lactam antibiotic analytes, wherein the beta-lactam moiety is disrupted by the reaction with the nucleophile derivatization reagent

### EXAMPLES

The following examples are provided to illustrate, not to limit the presently claimed invention.

### Example 1: Stability of Native Piperacillin

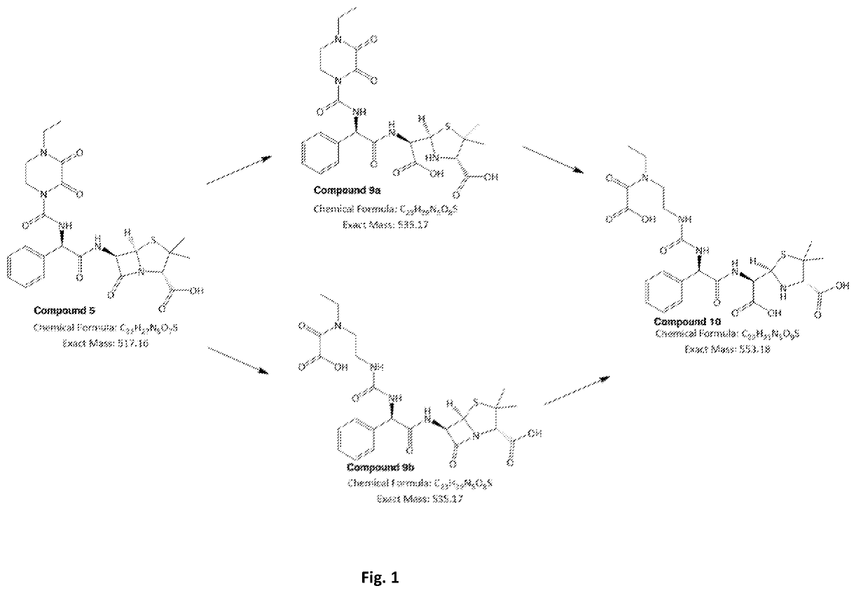

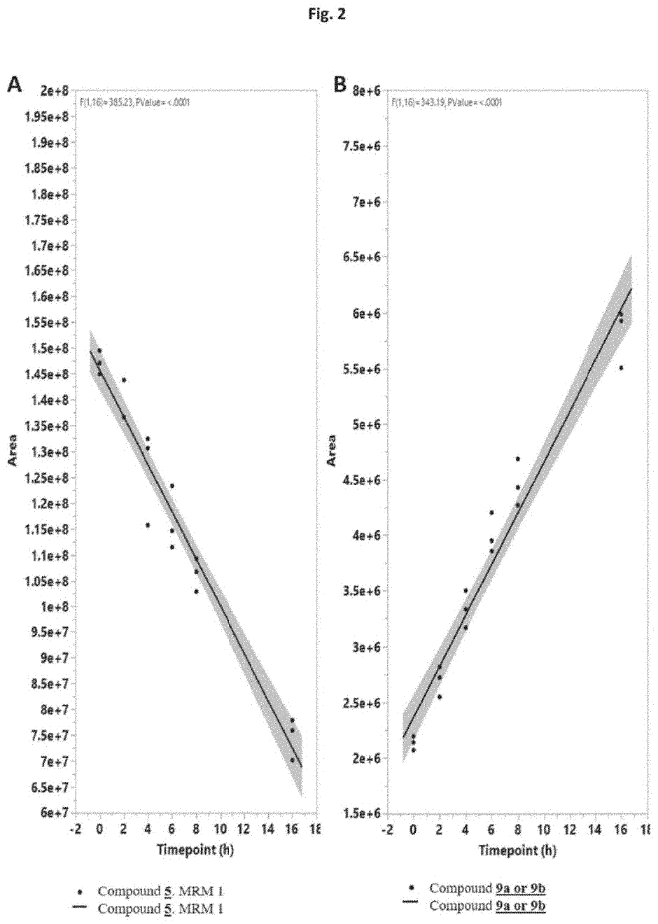

The stability of native Piperacillin as well as its hydrolyzed forms was investigated (compounds 5, 9a and 9b, respectively. From the hydrolyzation pathway of Piperacillin (see schematic drawing in FIG. 1) it is obvious that this compound hydrolyzed both on the piperazin ring and the lactam moiety, only one of both compounds is monitored to account for the loss of native Piperacillin). To this end, these compounds were freshly weighed and dissolved in water at a concentration of 1 mg/mL by rolling for 15 minutes at room temperature. Subsequently, these compounds were diluted to 5 μg/ml and measured with a suitable LC-MS/MS method at timepoints 0, 2, 4, 6, 8 and 16 h. For this, a Sunshell C18, 2.6 μm, 2.1 mm×50 mm column with Solvent A: water with 0.1% HCOOH and Solvent B: CH₃CN with 0.1% HCOOH and a flow of 0.6 mL per minute on an Agilent Infinity II multisampler/Pump system connected to an AB Sciex 6500+ MS was used. The peaks were integrated using MultiQuant software and the areas of these peaks depicted in the graphs in FIGS. 2A and 2B.

FIGS. 2A and 2B show the obtained areas for one MRM transitions for native Piperacillin (compound 5) and its hydrolyzed forms (compounds 9a/9b), respectively. It is clear that the obtained peak-areas vary significantly over time (F-test, yielding a P value of <0.0001) with the peak-areas of the native form decreasing and the peak-areas of the hydrolyzed forms (compounds 9a/9b) significantly increasing (F-test, yielding P value of <0.0001). The reason for this is the hydrolyzation (as is schematically demonstrated in FIG. 1).

### Example 2: Stability of Derivatized Piperacillin

To assess whether full β-Lactam derivatization is achievable using simple propylamine, butylamine or pentylamine, these nucleophiles were added in high excess to solutions of Meropenem and Piperacillin (1 μg/mL). For schematic drawing of the chemical reactions, see FIGS. 3 and 4, for Meropenem and Piperacillin, respectively.

The stability of double butylamide variants of Piperacillin (compound 7, see FIG. 4) was investigated at 2 MRMs using the identical protocol as in Examples 1.

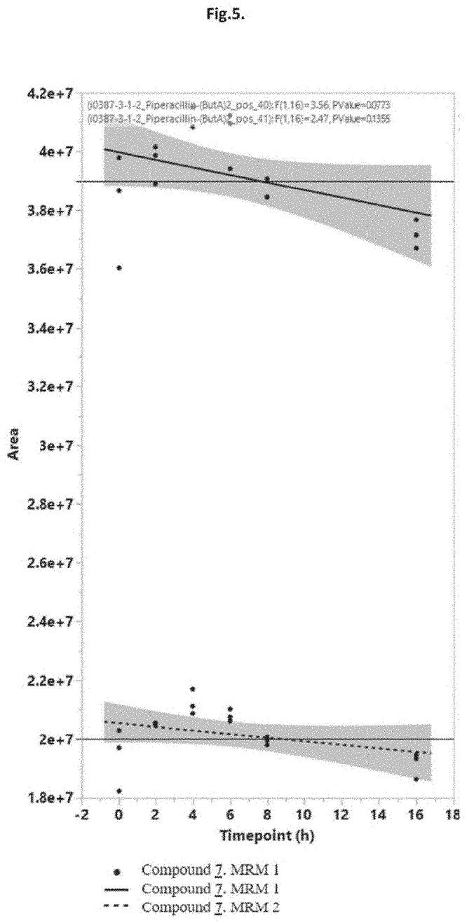

FIG. 5 shows the obtained areas for two MRM transitions for compound 7. It is observed that the obtained peak-areas do not vary significantly over time, i.e. the derivatized Piperacilin does not hydrolyse. This is further corroborated by a F-test, yielding P values of 0.08 and 0.14.

### Example 3: Stabilization of Meropenem and Piperacillin in Patient Samples

Derivatization reagents (propylamine, butylamine, or pentylamine), dissolved in water were added to 100 μL of sample (serum spiked with 1 μg/mL of both Piperacillin and Meropenem).

Relative to 100 μL of a 1 μg/mL (1.9*10⁻⁹M) Piperacillin, 5*10⁸, 2.5*10⁸ or 2.5*10⁶ equivalents (9.8*10⁻⁵, 4.8*10⁻⁵, 1.9*10⁻⁷ moles respectively) of the respective derivatization reagents (20 μL) were added to the spiked serum. This mixture was then incubated for 3 minutes after which a pH adjustment reagent (40 μL of an aqueous 1 M HCOOH (pH 2.5) or 500 mM Na₃PO₄/Na₂HPO₄ (pH 12)) was added. Subsequently, magnetic beads (40 μL, 50 mg/mL) were added and incubated for 3 minutes. The supernatant was next removed and the beads were washed twice with water (150 μL). Next, an elution solution (50 μL of a solution with either 100 mM HCOOH, 100 mM pyrrolidin or no pH adjustment reagent in varying levels of acetonitrile (10-90%, v/v) was added. The supernatant (20 μL) was next diluted with water (20 μL).

To quantify both the native (intact) Meropenem and Piperacillin, as well as their derivatized products and the hydrolyzed compounds, a LC-MS/MS method was devised including tuned MRM transitions for all compounds. A Cortecs C18+ C18, 2.6 μm, 2.1 mm×50 mm column with Solvent A; water with 0.1% HCOOH and Solvent B: CH₃CN with 0.1% HCOOH and a flow of 0.6 mL per minute on an Agilent Infinity II multisampler/Pump system connected to an AB Sciex 5500+ MS. For each of the derivatized antibiotics (i.e. Meropenem (a386) and Piperacillin (a0387), derivatized with either propylamine, butylamine or amylamine), three MRM transitions were used. Native Meropenem and Piperacillin, as well as their hydrolyzed forms (2 MRM transitions per analyte) were also included in the measurement.

| | Q1 | Q3 | | | | |
|---|---|---|---|---|---|---|
| | Mass | Mass | Time | DP | CE | CXP |
| Name | (Da) | (Da) | (min) | (volts) | (volts) | (volts) |
| a0386_Meropenem | 384.1 | 141 | 1.8 | 96 | 23 | 12 |
| a0387_Piperacillin_1 | 540.2 | 398 | 3.2 | 1 | 25 | 14 |
| a0386_PropA_pos1 | 443 | 181 | 1.7 | 31 | 31 | 6 |
| a0386_PropA_pos2 | 443 | 173 | 1.7 | 31 | 27 | 10 |
| a0386_PropA_pos3 | 443 | 399 | 1.7 | 26 | 15 | 20 |
| a0386_ButA_pos1 | 457.2 | 413.2 | 1.7 | 51 | 13 | 24 |
| a0386_ButA_pos2 | 457.2 | 195.2 | 1.7 | 51 | 25 | 16 |
| a0386_ButA_pos3 | 457.2 | 175.1 | 1.7 | 51 | 23 | 10 |
| a0386_PentA_pos1 | 471.2 | 427.2 | 1.7 | 56 | 15 | 20 |
| a0386_PentA_pos2 | 471.2 | 209.2 | 1.7 | 56 | 25 | 14 |
| a0386_PentA_pos3 | 471.2 | 175.1 | 1.7 | 56 | 25 | 10 |
| a0387_PropA_pos1 | 636.2 | 477.2 | 3.3 | 106 | 23 | 22 |
| a0387_PropA_pos2 | 636.2 | 361.2 | 3.3 | 106 | 27 | 16 |
| a0387_PropA_pos3 | 636.2 | 205 | 3.3 | 106 | 63 | 6 |
| a0387_ButA_pos1 | 664.2 | 375.1 | 3.3 | 126 | 27 | 24 |
| a0387_ButA_pos2 | 664.2 | 505.2 | 3.3 | 126 | 23 | 30 |
| a0387_ButA_pos3 | 664.2 | 106.1 | 3.3 | 126 | 39 | 14 |
| a0387_PentA_pos1 | 692.4 | 389.2 | 4.2 | 131 | 27 | 22 |
| a0387_PentA_pos2 | 692.4 | 533.3 | 4.2 | 131 | 25 | 28 |
| a0387_PentA_pos3 | 692.4 | 278.1 | 4.2 | 131 | 33 | 18 |
| a0386_Hydrolyzed | 399.8 | 152 | 1.8 | −60 | −24 | −5 |
| Meropenem_Neg_1 | | | | | | |
| a0386_Hydrolyzed | 399.8 | 173 | 1.8 | −60 | −34 | −13 |
| Meropenem_Neg_2 | | | | | | |
| a0387_Hydrolyzed | 551.9 | 270 | 1.8 | −30 | −34 | −15 |
| Piperacillin_Neg_1 | | | | | | |
| a0387_Hydrolyzed | 551.9 | 464 | 1.8 | −30 | −22 | −21 |
| Piperacillin_Neg_2 | | | | | | |

Q1 Mass = Quadrupole 1,
Q3 Mass = Quadrupole 3,
DP = Declustering Potential,
CE = Collision Energy,
CXP = Collision Cell Exit Potential

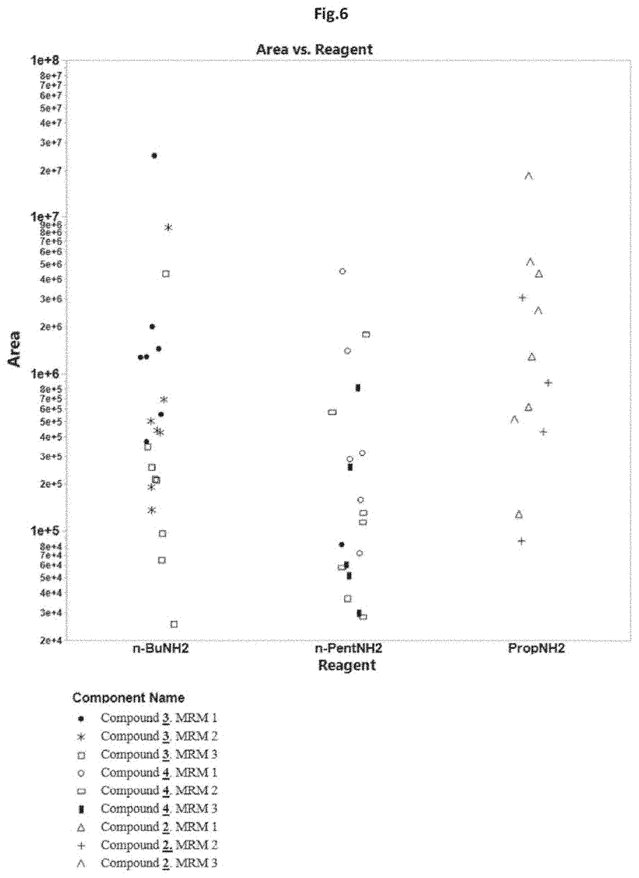

For Meropenem, the results with the highest peak-areas are obtained when using pentylamine. At a concentration of 1 μg/mL in serum of this antibiotic, using pentylamine and an optimal workflow, an area of about 3E6 should be possible. Using butylamine, under optimal conditions areas of 1E6 are achievable. See FIG. 6.

For Piperacillin the results with the highest peak-areas are obtained when using butylamine. At a concentration of 1 μg/mL in serum of this antibiotic, using butylamine and an optimal workflow (see next section for optimal workflows), an area of 2E7 should be possible. See FIG. 7.

It is noted, that no residual native compound (intact Meropenem or Piperacillin) is found in the eluate, when using 2.5E8 equivalents of the reagent. This shows that the reaction in this short time is quantitative. Furthermore, it was observed that no increase in the amount of hydrolyzed compound, indicating that the addition of the nucleophile does not catalyze hydrolyzation of the lactam moiety in these compounds, making it possible to discriminate and quantify the intact lactam compound from the hydrolyzed compound.

Example 3 shows that derivatization strategy works for two representative β-lactam antibiotics in combination with three different nucleophilic derivatization reagents, showing the validity and overall robustness of this method.

### Example 4 Degradation of Piperacillin in Serum

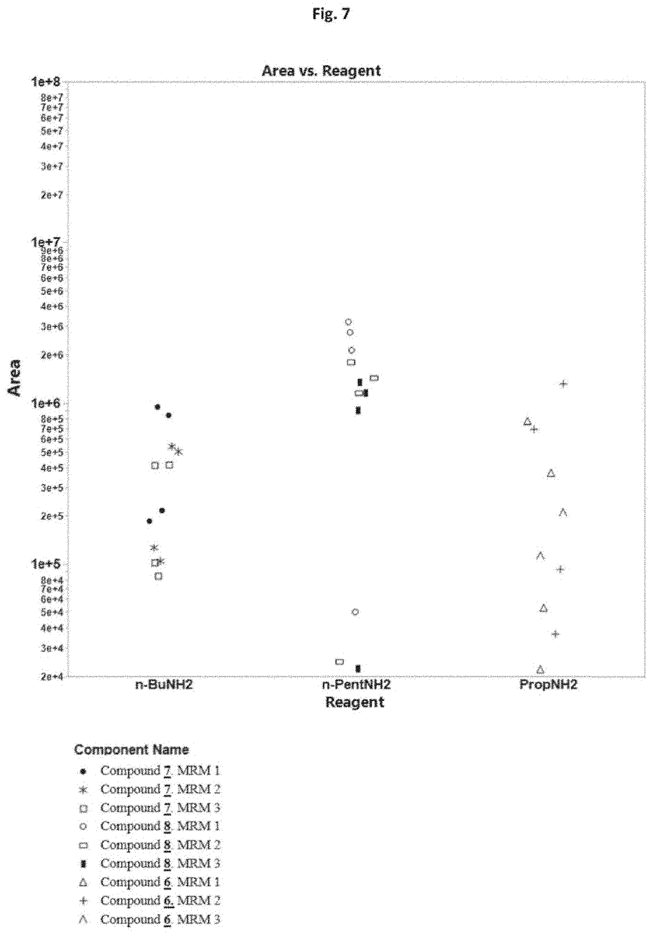

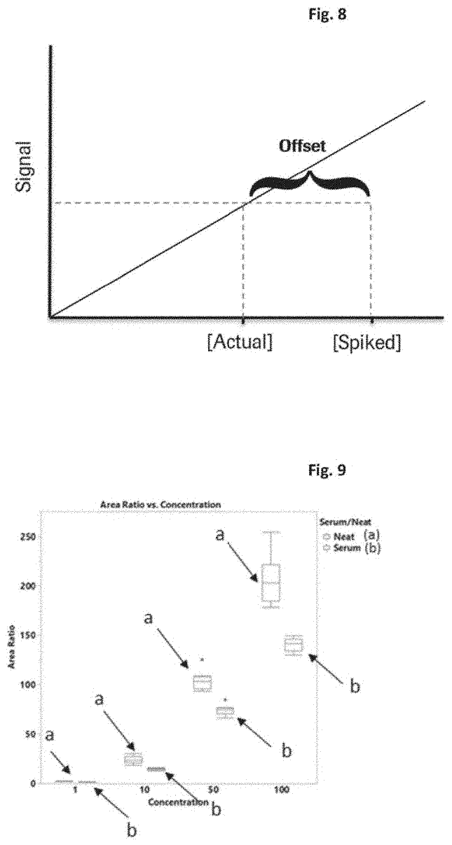

A major obstacle in the quantitation of this class of antibiotics is addressed in FIG. 8. As quantitation in general, be it via LC-MS/MS, UV or immune assays, relies on accurate calibration, it is obviously of utmost importance to use a reliable calibration method. However, as it can be shown here, β-Lactam antibiotics that are dissolved in serum are highly labile. The result of this is that the spiked concentration is higher than the actual concentration, leading to a calibration offset (see FIG. 8) that in turn results in inaccurate results.

Example 4 shows that If native β-lactam antibiotics are used for calibration purposes, these compounds degrade faster than the derivatized compounds proposed here. This means that calibration using native native β-lactam antibiotics yields inaccurate results. The use of stabilized (i.e. derivatized compounds) will for this reason yield more accurate results.

### Experimental Design

The inventors hypothesized, that β-Lactam antibiotics are more stable in a neat solution (i.e. water with 50% CH3CN) than in a serum based solution as the latter would offer a high concentration of nucleophilic substances that would hydrolyse or otherwise react with the β-Lactam moiety to obtain for example amides or esters. To test this assumption, piperacillin was dissolved in a solution of water/CH₃CN (1:1, v:v), which was then used to spike serum and the same solution of water/CH₃CN. Dissolution was performed only once, while spiking of this stock solution in serum or water/CH₃CN was performed three times for four different concentrations of piperacillin.

Prior to measurement, most methods in routine clinical diagnostics entail a purification workflow. Several methods can be used, ranging from protein precipitation using organic solvent followed by centrifugation to purification by means of magnetic beads. In case quantitation is performed via MS/MS, preferably an isotopically labeled internal standard (ISTD) is added at the beginning of this purification workflow to correct for i) analyte loss during this workflow and ii) ion suppression/enhancement that may differ between calibration samples and patient samples. It can be used an enrichment workflow in which the piperacillin is derivatized using butylamine to yield a dibutylamide (FIG. 4, compound 7, see Scheme below mentioned). Thereby, the β-Lactam moiety is reacted to a butylamide and the piperazin moiety reacts during this procedure. An ISTD that is a stable derivative of piperacillin, containing a single butylamide chain and a D5-labeling on the phenyl moiety is preferably added. This ISTD therefore is not subject to nucleophilic substitution that leads to disintegration of the β-Lactam moiety. However, during the workflow, the second amidation that takes place on the piperazin ring also takes place (see Scheme below mentioned). Thus, although more stable by nature, the ISTD will not disintegrate as fast as the native piperacillin, the amidation on the piperazin ring is an in-line control that ascertains that amidation using butylamine works.

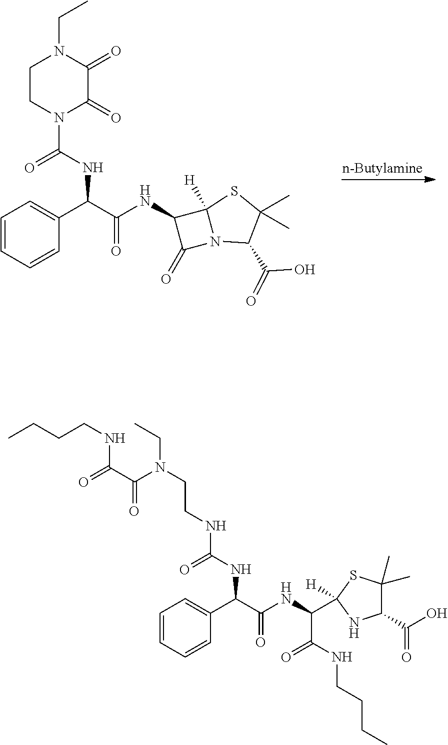 [SMILES: CCN1CCN(C(=O)N[C@@H](C(=O)N[C@@H]2C(=O)N3[C@@H]2SC(C)(C)[C@@H]3C(=O)O)c2ccccc2)C(=O)C1=O ; CCCCNC(=O)C(=O)N(CC)CCNC(=O)N[C@@H](C(=O)N[C@H](C(=O)NCCCC)[C@@H]1N[C@@H](C(=O)O)C(C)(C)S1)c1ccccc1]

Piperacillin is derivatized using butylamine to yield a dibutylamide

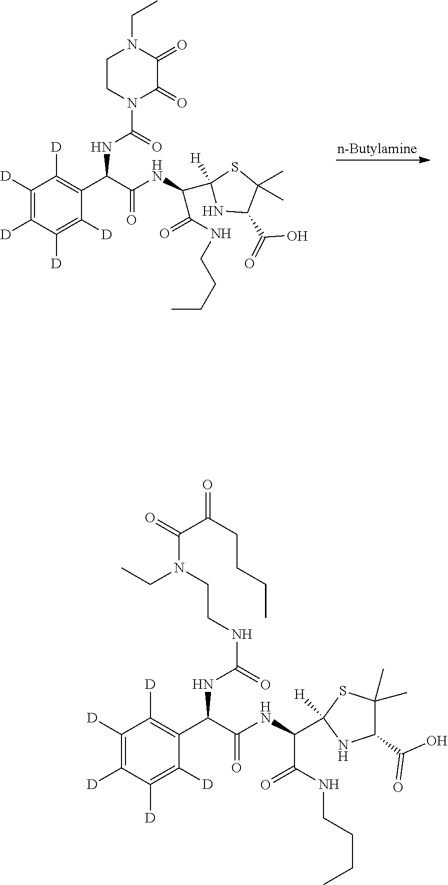 [SMILES: [2H]c1c([2H])c([2H])c([C@@H](NC(=O)N2CCN(CC)C(=O)C2=O)C(=O)N[C@H](C(=O)NCCCC)[C@@H]2N[C@@H](C(=O)O)C(C)(C)S2)c([2H])c1[2H] ; [2H]c1c([2H])c([2H])c([C@@H](NC(=O)NCCN(CC)C(=O)C(=O)CCCC)C(=O)N[C@H](C(=O)NCCCC)[C@@H]2N[C@@H](C(=O)O)C(C)(C)S2)c([2H])c1[2H]]

Piperacillin-butylamide-D5 is derivatized using butylamine to yield Piperacillin-dibutylamide-D5

### Materials and Methods

### Material

Piperacillin was obtained from Sigma Aldrich.

Quality Control materials were from Chromsystems and following dissolution concentrations of 19.2 and 97.9 μg/mL were obtained.

### Methods

### Weighing and Spiking

Piperacillin was weighed and dissolved directly into water/CH₃CN (1:1, v:v) to obtain a concentration of 1 mg/mL. This stock solution was then used to spike either serum pool or water/CH₃CN (1:1, v:v) to obtain concentrations of 1, 10, 50 and 100 μg/mL. This spiking was repeated three times for each concentration.

Subsequently, ail samples were homogenized for 20 min. by rolling. Next, the samples were placed at a sample preparation module, where each sample is processed as described in the following section.

### Sample Preparation

Preferably, ISTD (piperacillin-butylamide-D5, 20 μg/mL, 20 μL) was added to a spiked serum or neat solution (50 μL). To this mixture, n-butylamine (5M, 50 μL) was added. This mixture was first shaken and incubated for 3 min at room temperature (rt). Next, magnetic beads (beadtype B, 50 mg/mL, 40 μL) were added, after which the mixture was shaken again and incubated for about 1 min. Subsequently, the beads were pulled to the side of the vessel by applying magnetic force, after which the supernatant was removed. These beads were washed twice with water (150 μL). Next, acetonitrile with 0.1% HCOOH (50 μL) was added, after which the mixture was shaken again and left to stand for 1 min. Next, the beads were pulled to the side of the vessel, after which 20 μL of supernatant was removed. This supernatant was then diluted with water (1:1, v:v), after which the samples were measured via LC-MS/MS.

### LC-MS/MS Measurements

To quantify the two-fold derivatized piperacillin derivatives LC-MS/MS methods were developed. The next table shows which fragments under which setting were used for this purpose.

| | | Time | | | | |
|---|---|---|---|---|---|---|
| Q1 | Q3 | (msec) | ID | DP | CE | CXP |
| 536.159 | 350.9 | 5 | Piperacillin-hydrolysed_{—} | 131 | 25 | 6 |
| 518.3 | 143 | 5 | native_Piperacillin | 91 | 27 | 14 |
| 591.152 | 143 | 5 | Piperacillin-(Butylamide)_pos_01 | 11 | 31 | 22 |
| 591.152 | 449.1 | 5 | Piperacillin-(Butylamide)_pos_02 | 11 | 25 | 38 |
| 664.235 | 375.1 | 5 | Piperacillin- | 126 | 27 | 24 |
| | | | (Dibutylamide)_pos_01 | | | |
| 664.235 | 505.2 | 5 | Piperacillin- | 126 | 23 | 30 |
| | | | (Dibutylamide)_pos_02 | | | |
| 664.235 | 106.1 | 5 | Piperacillin- | 126 | 39 | 14 |
| | | | (Dibutylamide)_pos_03 | | | |
| 596.25 | 143.167 | 5 | Piperacillin-D5- | 171 | 27 | 20 |
| | | | (butylamide)_pos_01 | | | |
| 596.25 | 114.09 | 5 | Piperacillin-D5- | 171 | 79 | 14 |
| | | | (butylamide)_pos_02 | | | |
| 596.25 | 115.25 | 5 | Piperacillin-D5- | 171 | 107 | 18 |
| | | | (butylamide)_pos_03 | | | |
| 669.235 | 380.1 | 5 | Piperacillin-D5- | 126 | 27 | 24 |
| | | | (dibutylamide)_pos_01 | | | |
| 669.235 | 510.2 | 5 | Piperacillin-D5- | 126 | 23 | 30 |
| | | | (dibutylamide)_pos_02 | | | |
| 669.235 | 111.1 | 5 | Piperacillin-D5- | 126 | 39 | 14 |
| | | | (dibutylamide)_pos_03 | | | |

### LC Method

| Time | % B |
|---|---|
| 0 | 1 |
| 4 | 50 |
| 4.5 | 98 |
| 5 | 1 |
| 6 | 1 |

A Kinetex C18, 2,6 μm, 1.0 mm×50 mm column with Solvent A: water with 0.1% HCOOH and Solvent B: CH₃CN with 0.1% HCOOH and a flow of 0.4 mL per minute on an Agilent Infinity II multisampler/pump system connected to an AB Sciex 6500+ MS, injecting 8 μL per sample.

### Results

FIG. 9 shows the difference in area ratio between samples in neat and from serum for four concentrations. For each concentration, it is shown that this difference is about 30%. The differences in area ration (for which an internal standard can be used), cannot be attributed to a difference in analyte recovery that is different for sample preparation of samples in neat vs, serum samples. The internal standard would be compensating for this effect. Therefore, the difference is most likely be due to the reactivity of the compound. As serum contains many reactive nucleophiles that are able to react with either the lactam or the piperazin moiety, the spiked concentration decreases over time in this matrix relative to a same concentration spiked in neat.

This finding may have implications in the quantitation of these antibiotics as the spiked concentration is higher than the actual concentration, which is a function of time, temperature, protein concentration or concentration of other nucleophilic substances. Therefore, the use of native piperacillin as spiking material to prepare calibration standards is likely to fail. This shows again, that quantitation of these analytes via the here described derivatization method will be more accurate.

### Example 5: Comparison Routinely Used Hospital Method vs. Derivatization Method for Piperacillin

To ensure longtime stability and accurate and precise quantitation of β-lactam antibiotics, the inventors envisage a strategy that makes use of a derivatization of this class of antibiotics. This also entails the use of pre-derivatized calibrators and ISTDs. Following in-house assay development, an experiment was conducted whereby commercial QC samples that are routinely used in at east one hospital, e.g. a German hospital, were used. To assess how the derivatization method deviates from the method that is routinely used in the hospital, 23 patient samples were collected and measured using both methods.

Example 5 shows that the here presented derivatization method correlates well with a routine method, however a difference of on average 20% in accuracy is observed between the two methods. This offset in accuracy is explained in example 4.

### Materials and Methods

### Materials

### Calibration Materials Used for Derivatization Strategy

Single derivatized piperacillin (piperacillin-butylamide) i387-2-2 was weighed and spiked in powder form directly in serum from which further dilutions were prepared to yield the calibration series in the following table.

| | | Concentration | |
|---|---|---|---|
| | Concentration | Piperacillin- | Concentration |
| | Piperacillin- | Butylamide | Piperacillin- |
| | Butylamide | trihydrate | Butylamide |
| | (ng/mL) | (μM) | (μM) |
| Calibrators for | 477.10 | 0.745287 | 0.808307 |
| Derivatization method | | | |
| Calibrators for | 596.38 | 0.931609 | 1.010383 |
| Derivatization method | | | |
| Calibrators for | 715.65 | 1.117931 | 1.21246 |
| Derivatization method | | | |
| Calibrators for | 1192.76 | 1.863218 | 2.020767 |
| Derivatization method | | | |
| Calibrators for | 5963.79 | 9.316091 | 10.10383 |
| Derivatization method | | | |
| Calibrators for | 59637.89 | 93.16091 | 101.0383 |
| Derivatization method | | | |
| Calibrators for | 119275.77 | 186.3218 | 202.0767 |
| Derivatization method | | | |
| Calibrators for | 238551.54 | 372.6436 | 404.1534 |
| Derivatization method | | | |

### Calibration Materials Used for Hospital Method

| | |
|---|---|
| Concentration | |
| Piperacillin (ng/mL) | Concentration Piperacillin (μM) |

| | | |
|---|---|---|
| Cal 1 | 4050 | 7.830929 |
| Cal 2 | 30130 | 58.25825 |
| Cal 3 | 60010 | 116.0331 |
| Cal 4 | 90150 | 174.3107 |
| Cal 5 | 120030 | 232.0855 |
| Cal 6 | 149910 | 289.8604 |
| Cal 7 | 174980 | 338.3348 |
| Cal 8 | 200050 | 386.8092 |

### Quality Controls Used for Hospital Method

| | | Concentration | Concentration |
|---|---|---|---|
| Origin | | Piperacilli (ng/mL) | Piperacilli (μM) |
| Calibrators Hospital | Cal 1 | 4050 | 7.83093 |
| Calibrators Hospital | Cal 2 | 30130 | 58.2583 |
| Calibrators Hospital | Cal 3 | 60010 | 116.033 |
| Calibrators Hospital | Cal 4 | 90150 | 174.311 |
| Calibrators Hospital | Cal 5 | 120030 | 232.086 |
| Calibrators Hospital | Cal 6 | 149910 | 289.86 |
| Calibrators Hospital | Cal 7 | 174980 | 338.335 |
| Calibrators Hospital | Cal 8 | 200050 | 386.809 |

### Quality Controls Used for Derivatization Method and for Hospital Method

| | Concentration | Concentration |
|---|---|---|
| | (ng/mL) | (μM) |
| QC Level I | 19200.00 | 37.12441 |
| QC Level II | 97900.00 | 189.2958 |

### Patient Samples Containing Piperacillin

| | Concentration | Concentration |
|---|---|---|
| Sample Name | (ng/mL) | (μM) |
| Clinical sample-15 | 8150 | 15.75854 |
| Clinical sample-32 | 9770 | 18.89091 |
| Clinical sample-36 | 343000 | 663.212 |
| Clinical sample-42 | 80500 | 155.6518 |
| Clinical sample-54 | 21500 | 41.5716 |
| Clinical sample-58 | 19600 | 37.89783 |
| Clinical sample-61 | 24100 | 46.59886 |
| Clinical sample-62 | 45800 | 88.55718 |
| Clinical sample-71 | 33300 | 64.38764 |
| Clinical sample-78 | 49900 | 96.48478 |
| Clinical sample-81 | 86000 | 166.2864 |
| Clinical sample-85 | 32100 | 62.06737 |
| Clinical sample-88 | 103000 | 199.157 |
| Clinical sample-90 | 20500 | 39.63804 |
| Clinical sample-96 | 14200 | 27.45659 |
| Clinical sample-98 | 25300 | 48.91914 |
| Clinical sample-104 | 19700 | 38.09119 |
| Clinical sample-114 | 13900 | 26.87652 |
| Clinical sample-117 | 15400 | 29.77687 |
| Clinical sample-121 | 24000 | 46.40551 |
| Clinical sample-125 | 6150 | 11.89141 |
| Clinical sample-133 | 40800 | 78.88936 |
| Clinical sample-134 | 33800 | 65.35442 |
| Clinical sample-137 | 52900 | 102.2855 |

### Methods

### Sample Preparation for Derivatization Strategy

Preferably, to either calibration sample, QC sample or patient sample (50 μL) was added ISTD (piperacillin-butylamide-D5, 20 μg/mL, 20 μL). To this mixture, n-butylamine (5M, 50 μL) was added. This mixture was first shaken and incubated for min at rt. Next, magnetic beads (beadtype B, 50 mg/mL, 40 μL) were added, after which the mixture was shaken again and incubated for about 1 min. Subsequently, the beads were pulled to the side of the vessel by applying magnetic force, after which the supernatant was removed. These beads were washed twice with water (150 μL). Next, acetonitrile with 0.1% HCOOH (50 μL) was added, after which the mixture was shaken again and left to stand for 1 min. Next, the beads were pulled to the side of the vessel, after which 20 μL of supernatant was removed. This supernatant was then diluted with water (1:1, v:v), after which the samples were measured via LC-MS/MS. All clinical patient samples were processed one after the other in a non-randomized fashion. Therefore, a time difference of about 90 min. exists between the processing of sample 1 and 23. A time difference of about 4 existed between the measurement of the three replicates that were processed for each sample.

### Sample Preparation for Hospital Method

Preferably, to either calibration sample, QC sample or patient sample (50 μL) was added ISTD (piperacillin-DS, 100 μg/mL, 25 μL). This mixture was vortexed shortly and shaken for 5 min. Subsequently MeOH (325 μL) was added and vortexed shortly and shaken for 5 min. Next, the vials were centrifuged (14000 rpm at 5° C.) and the supernatant (20 μL) was diluted with water (180 μL). These solutions were measured via LC-MS/MS. All clinical patient samples were processed one after the other in a non-randomized fashion.

### LC-MS/MS Measurements for Derivatization Method

To quantify the two-fold derivatized piperacillin derivatives LC-MS/MS methods were developed. The next table shows which fragments under which setting were used for this purpose.

| | | Time | | | | |
|---|---|---|---|---|---|---|
| Q1 | Q3 | (msec) | ID | DP | CE | CXP |
| 536.159 | 350.9 | 5 | Piperacillin-hydrolysed_{—} | 131 | 25 | 6 |
| 518.3 | 143 | 5 | native_Piperacillin | 91 | 27 | 14 |
| 591.152 | 143 | 5 | Piperacillin- | 11 | 31 | 22 |
| | | | (Butylamide)_pos_01 | | | |
| 591.152 | 449.1 | 5 | Piperacillin- | 11 | 25 | 38 |
| | | | (Butylamide)_pos_02 | | | |
| 664.235 | 375.1 | 5 | Piperacillin- | 126 | 27 | 24 |
| | | | (Dibutylamide)_pos_01 | | | |
| 664.235 | 505.2 | 5 | Piperacillin- | 126 | 23 | 30 |
| | | | (Dibutylamide)_pos_02 | | | |
| 664.235 | 106.1 | 5 | Piperacillin- | 126 | 39 | 14 |
| | | | (Dibutylamide)_pos_03 | | | |
| 596.25 | 143.167 | 5 | Piperacillin-D5- | 171 | 27 | 20 |
| | | | (butylamide)_pos_01 | | | |
| 596.25 | 114.09 | 5 | Piperacillin-D5- | 171 | 79 | 14 |
| | | | (butylamide)_pos_02 | | | |
| 596.25 | 115.25 | 5 | Piperacillin-D5- | 171 | 107 | 18 |
| | | | (butylamide)_pos_03 | | | |
| 669.235 | 380.1 | 5 | Piperacillin-D5- | 126 | 27 | 24 |
| | | | (dibutylamide)_pos_01 | | | |
| 669.235 | 510.2 | 5 | Piperacillin-D5- | 126 | 23 | 30 |
| | | | (dibutylamide)_pos_02 | | | |
| 669.235 | 111.1 | 5 | Piperacillin-D5- | 126 | 39 | 14 |
| | | | (dibutylamide)_pos_03 | | | |

### LC Method for Derivatization Method

| Time | % B |
|---|---|
| 0 | 1 |
| 4 | 50 |
| 4.5 | 98 |
| 5 | 1 |
| 6 | 1 |

A Kinetex C18, 2.6 μm, 1.0 mm×50 mm column with Solvent A: water with 0.1% HCOOH and Solvent B: CH₃CN with 0.1% HCOOH and a flow of 0.4 mL per minute on an Agilent Infinity II multisampler/pump system connected to an AB Sciex 6500+ MS, injecting 8 μL per sample.

### LC-MS/MS Measurements for Hospital Method

| Q1 Mass | Q3 Mass | | | | | |
|---|---|---|---|---|---|---|
| (Da) | (Da) | Time | ID | DP | CE | CXP |
| 518.2 | 359.2 | 75 | Piperacillin_Quan | 25 | 11 | 19 |
| 518.2 | 143.1 | 75 | Piperacillin_Qual | 25 | 25 | 19 |
| 523.1 | 364.1 | 25 | Piperacillin- | 25 | 11 | 19 |
| | | | d5_Quan | | | |
| 536.2 | 492.2 | 75 | Hydrolysed- | 45 | 20 | 18 |
| | | | Piperacillin_Quan | | | |
| 536.2 | 350.1 | 75 | Hydrolysed- | 45 | 25 | 18 |
| | | | Piperacillin_Qual | | | |
| 541.2 | 497.2 | 25 | Hydrolysed- | 45 | 20 | 18 |
| | | | Piperacillin- | | | |
| | | | d5_Quan | | | |
| 554.2 | 510.1 | 75 | Bis-Hydrolysed- | 45 | 16 | 20 |
| | | | Piperacillin_Quan | | | |
| 559.3 | 515.1 | 25 | Bis-Hydrolysed- | 45 | 16 | 20 |
| | | | Piperacillin- | | | |
| | | | d5_Quan | | | |

### LC Method for Hospital Method

| Time [min] | [%] B |
|---|---|
| 0 | 7 |
| 1 | 7 |
| 1.01 | 80 |
| 1.8 | 100 |
| 2.5 | 100 |
| 2.51 | 20 |
| 3.25 | 100 |
| 5 | 100 |
| 5.25 | 7 |
| 5.5 | 7 |

A XSelect HSS PFP 2.5 μm (2.1×100 mm) column with a XSelect HSS PFP Van Guard Cartridge (2.1×5 mm) from Waters was used. Solvent A: water with 10 mM ammonium formiate with 0.2% formic acid, and Solvent B: CH₃CN/MeOH (25:75, v:v) with a flow of 0.5 mL per minute on an Agilent Infinity II multisampler/pump system connected to an AB Sciex 6500+ MS, injecting 2 μL per sample.

### Results

### Precision and Accuracy

While precision may be calculated from the variance of the obtained results, accuracy can only be determined given the right or theoretical concentration. As it has established previously in example 4, the correct concentration is not equal to the spiked concentration, but a concentration that is below this concentration. Nevertheless, to be able to calculate the difference between the derivatization method described here and a reference method, it is made the assumption that the spiked concentration equals actual concentration, being aware that this is not correct. However, as a relative measure of accuracy, this is still a useful indicator.

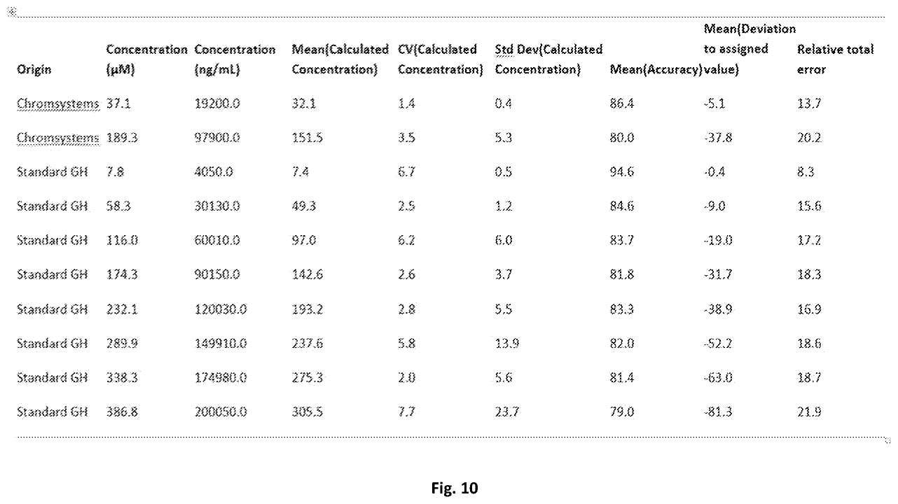

It can be seen that precision in terms of CV is very low for the quality control samples with CV's of less than 4%. The accuracy of these samples is 86.4 and 80%. Again, this is based on the assumption that the spiked concentrations of the calibrators as used in the reference method are equal to the actual concentration. However, the real accuracy shall be closer to 100%. In addition, since the relative total error is calculated based on accuracy, this error shall be closer to 0 than the values calculated in FIG. 10.

### Correlation Between Methods

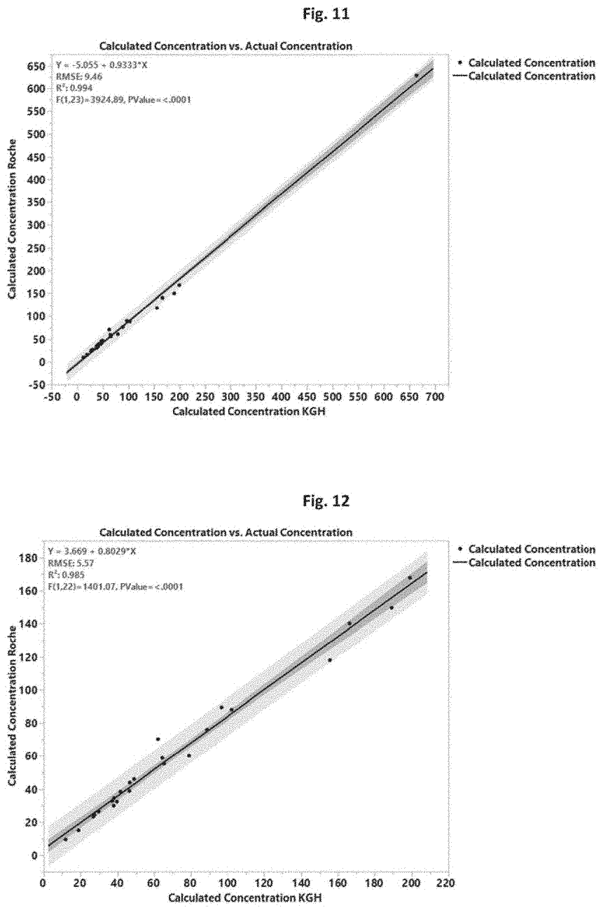

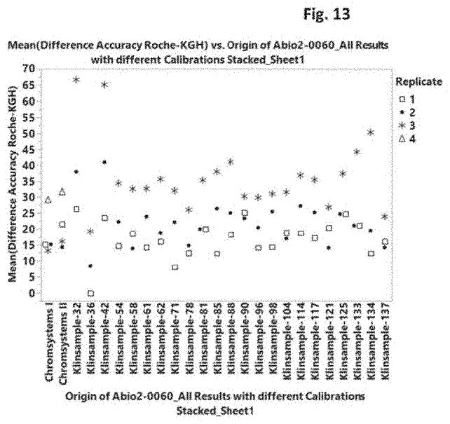

To see how both evaluated methods relate FIGS. 11 and 12 were produced using JMP version 14.3. included in the analysis are R2 and a F-test that show high correlation between the two methods. FIG. 11 shows the correlation calculated concentrations from both methods, wherein all samples are included. FIG. 12 shows the correlation calculated concentrations from both methods, wherein the highest concentrated sample is excluded for clarity. FIG. 13 shows the difference in accuracy between the two methods per replicate. I.e. (Accuracy derivatization method)−(accuracy hospital method).

All samples that were processed with a derivatization method were processed three times, with a time lapse of about 4 hours between replicate 1 and 3, in which the samples were left to stand at the pipetting robot at a temperature between 25 and 30° C. The implication of this time difference is clearly visible in FIG. 13. Here, it can be seen that the difference in accuracy between the two methods is smallest for replicate 1, whereas replicates 2 and 3 show a much greater deviation of the original value. Since the degradation of this analyte over time is substantial, the low accuracy of replicates 2 and 3 area consequence of this. This also entails, that any attempt to calculate a CV from these values is pointless, as it would be far greater than what the method itself is capable of. Nevertheless, the interesting result from this is that, although all replicates of a single sample have the same time-lapse between them, the difference in calculated concentrations is variable for all samples. For example, clinical sample 42 shows around 40% degradation over 4 hours, while clinical sample 137 only shows about 10% degradation over the same time period. The finding that different clinical samples show differences in decreasing piperacillin concentrations suggest that the different clinical samples display different degradation kinetics for piperacillin. This means that it is absolutely pivotal to process and measure a patient sample as soon as possible after the sample is obtained. More so, this also shows once more that routinely used methods that make use of calibration materials that contain spiked piperacillin and an ISTD that is an isotopically labeled variant of piperacillin, are likely to lead to an inaccurate result that is an overestimation of the true value.

This patent application claims the priority of the European patent application 19209516.4, wherein the content of this European patent application is hereby incorporated by references.

1) A method of determining the amount or concentration of one or more derivatized antibiotic analytes in an obtained sample comprising
a) pre-treating and/or enriching the sample by using magnetic beads, and
b) determining the amount or concentration of the one or more antibiotic analytes in the sample by using immunological assay or LC/MS,
wherein the one or more antibiotic analytes are derivatized with a nucleophilic derivatization reagent to stabilize the one or more antibiotic analytes.

2) The method of claim 1, wherein the antibiotic analyte is Piperacillin.

3) The method of claim 1, wherein the antibiotic analyte is Meropenem.

4) The method of claim 1, wherein the nucleophilic derivatization reagent is selected from the group consisting of propylamine, butylamine, and pentylamine.

5) A method of determining the amount or concentration of one or more antibiotic analytes in an obtained sample, comprising
a) pre-treating the sample with a derivatization reagent, wherein the derivatization reagent comprises a nucleophile to stabilize the one or more antibiotic analytes,
b) enriching the sample obtained after step a) by using magnet beads, and
c) determining the amount or concentration of the one or more antibiotic analytes in the pre-treated sample obtained after step a) or after the enrichment step b) by using immunological assay or LC/MS.

6) The method of claim 5, wherein the antibiotic analyte is a β-lactam antibiotic analyte.

7) The method of claim 5, wherein the antibiotic analyte is Meropenem or Piperacillin.

8) The method of claim 5, wherein in step a) the sample is pre-treated with a nucleophilic derivatization reagent immediately after the sample is obtained.

9) The method of claim 5, wherein the sample obtained after step a) comprises antibiotic analytes derivatized with a nucleophilic derivatization reagent.

10) The method of claim 5, wherein enrichment step b) comprises at least one enrichment workflow.

11) (canceled)

12) A sampling tube for collecting a patient sample comprising a device with a reservoir adapted for receiving a blood sample to be collected, and a nucleophilic derivatization reagent suitable to stabilize one or more antibiotic analytes in a sample.

13) (canceled)

14) (canceled)

15) (canceled)

16) The method of claim 4, wherein the butylamine is primary linear butylamine.

17) The method of claim 8, wherein the sample is pre-treated with a nucleophilic derivatization reagent within a time period selected from within less than 10 min after the sample is obtained or within less than 5 min after the sample is obtained.

18) The method of claim 1, wherein the nucleophilic derivatization reagent prevents hydrolyzation of the antibiotic during determining step b).

19) The method of claim 5, wherein the nucleophilic derivatization reagent prevents hydrolyzation of the antibiotic during determining step c).

20) The method of claim 18, wherein the nucleophilic derivatization reagent stabilizes the antibiotic by forming a covalent adduct of the one or more antibiotic analytes and the nucleophilic derivatization reagent.

21) The method of claim 19, wherein the nucleophilic derivatization reagent stabilizes the antibiotic by forming a covalent adduct of the one or more antibiotic analytes and the nucleophilic derivatization reagent.

22) The method of claim 20, wherein the nucleophilic derivatization reagent stabilizes the antibiotic for more than 2 hours, for more than 4 hours, for more than 8 hours, for more than 12 hours, for more than 15 hours, for more than 24 hours, for more than 48 hours, for more than 7 days, for more than 2 weeks, for more than 4 weeks, for more than 2 months, for more than 3 months, for more than 4 months, for more than 5 months, or for more than 6 months.
